(19) United States
(12) Patent Application Publication
(10) Pub. No.: US 2013/0212741 A1
(43) Pub. Date: Aug. 15, 2013

(54) MOLECULAR CLONING OF BROWN-MIDRIB2 (bm2) GENE

(71) Applicant: Iowa State University Research Foundation, Inc., (US)

(72) Inventors: Patrick S. Schnable, Ames, IA (US); Ho Man Tang, Ames, IA (US); Sanzhen Liu, Ames, IA (US); Wei Wu, Johnston, IA (US)

(73) Assignee: IOWA STATE UNIVERSITY RESEARCH FOUNDATION, INC., Ames, IA (US)

(21) Appl. No.: 13/751,829

(22) Filed: Jan. 28, 2013

Related U.S. Application Data
(60) Provisional application No. 61/592,379, filed on Jan. 30, 2012.

(51) Int. Cl. C12N 15/82

(52) U.S. Cl. CPC C12N 15/8255
USPC 800/286; 800/278; 800/298; 506/2; 800/285

(57) ABSTRACT

The present invention relates to methods for altering the concentration or composition of lignin in a plant; a method of making a mutant plant having an altered level of MTHFR protein compared to that of a nonmutant plant; plants, mutant plants, and mutant plant seeds produced by these methods; a method of identifying a candidate plant suitable for breeding that displays an altered lignin concentration or composition phenotype; a transgenic plant having an altered level of MTHFR protein capable of determining the lignin concentration or composition in a plant compared to that of a nontransgenic plant; and seed produced from a transgenic plant.

This application claims the benefit of U.S. Provisional Patent Application Ser. No. 61/592,379, filed Jan. 30, 2012, which is hereby incorporated by reference in its entirety.

This invention was made with government support under grant numbers DEB0919348, IOS0820610, and DBI0527192 awarded by the National Science Foundation. The government has certain rights in the invention.

### FIELD OF THE INVENTION

The present invention relates to the molecular characterization of the bm2 gene in plants, methods of altering lignin concentration or composition in plants, and plants with altered lignin concentration or composition.

### BACKGROUND OF THE INVENTION

Lignin is a heterogeneous aromatic polymer that serves as a major component of cell walls. It plays a critical role in the structural integrity of vascular plants (Sarkanen & Ludwig, Definition and Nomenclature. In Lignins: Occurrence, Formation, Structure and Reactions, Wiley-Interscience, New York, 1971). In lignified tissues, lignin is heavily cross-linked with cellulose and hemicelluloses such that it provides protects and strengthens the tissues, and also increases the resistance of biomass to the enzymatic digestion of its components by ruminants as well as by bacteria and fungi (Sarkanen & Ludwig, Definition and Nomenclature. In Lignins: Occurrence, Formation, Structure and Reactions, Wiley-Interscience, New York, 1971). Because lignin has this high level of resistance to enzymatic digestion, lignin has a negative impact on forage quality and cellulosic bio fuel production (Penning et al., “Genetic Resources for Maize Cell Wall Biology,” Plant Physiol. 151:1703-1728 (2009); Ragauskas et al., “The Path Forward for Bio fuels and Biomaterials,” Science 311:484-489 (2006)). In contrast, a reduction in lignin content significantly improves digestibility and animal performance. Reduced lignin content of livestock feed also protects the environment against excessive animal waste (Jung et al., “Influence of Lignin on Digestibility of Forage Cell Wall Material,” J. Animal Sci. 62:1703-1712 (1986)). High levels of enzyme-resistant lignin also results in recalcitrance of sugar release for fermentation mediated by microorganisms so that this is a major limitation for conversion of lignocellulosic biomass to biofuel such as ethanol (Fu et al., “Genetic Manipulation of Lignin Reduces Recalcitrance and Improves Ethanol Production from Switchgrass,” Proc. Natl. Acad. Sci. USA 108:3803-3808 (2011)). Therefore, understanding the mechanism mediating the lignin content in crops will provide important insights into the improvement of crops and forage quality as well as biofuel production.

Lignin is composed of three hydroxycinnamyl alcohol subunits (monolignols), p-coumaryl, coniferyl, and sinapyl alcohol, resulting in hydroxyphenyl (H), guaiacyl (G) and syringyl (S) types of lignin, respectively (Bonawitz et al., “The Genetics of Lignin Biosynthesis: Connecting Genotype to Phenotype,” Ann. Rev. Gen. 44:337-363 (2010); Whetten et al., “Lignin Biosynthesis,” Plant Cell 7:1001-1013 (1995)). Monolignols are synthesized by enzymes in the phenylpropanoid pathway, including cinnamyl alcohol dehydrogenase (CAD), and cinnamoyl CoA-reductase (CCR) (Lewis et al., “Lignin: Occurrence, Biogenesis and Biodegradation,” Ann. Rev. Plant Physiol. Plant Mol. Biol. 41:455-496 (1990); Tamasloukht et al., “Characterization of a Cinnamoyl-CoA Reductase 1 (CCR1) Mutant in Maize: Effects on Lignification, Fibre Development, and Global Gene Expression,” J. Exp. Bot. 62:3837-3848 (2011)). Para-coumaric acid, a major component of lignocellulose, is synthesized from cinnamic acid by the action of the P450-dependent enzyme 4-cinnamic acid hydroxylase (Boerjan et al., “Lignin Biosynthesis,” Annual Review of Plant Biology 54: 519-546 (2003). O-methyltransferases (OMT) converts para-coumaric acid to ferulic and sinapic acid (Tu et al., “Functional Analyses of Caffeic Acid O-Methyltransferase and Cinnamoyl-CoA-reductase Genes from Perennial Ryegrass (Lolium perenne),” Plant Cell 22:3357-3373 (2010); Whetten et al., “Lignin Biosynthesis,” Plant Cell 7:1001-1013 (1995)). Para-coumaric acid (PCA), ferulic acid (FA) and sinapic acid are converted to monolignols p-coumaric, coniferyl, and syringul alcohol, which are further converted to the H, G, and S types of lignin (Bonawitz et al., “The Genetics of Lignin Biosynthesis: Connecting Genotype to Phenotype,” Ann. Rev. Gen. 44:337-363 (2010); Whetten et al., “Lignin Biosynthesis,” Plant Cell 7:1001-1013 (1995)). However, the molecular mechanisms of lignin biosynthesis have not yet been fully elucidated.

Maize (Zea mays ssp. mays L.) is one of the most wildly grown and most highly productive crops that contribute to the production of food, feed, and bio fuels (Doebley et al., “The Molecular Genetics of Crop Domestication,” Cell 127:1309-1321 (2006); Tang et al., “Domestication and Plant Genomes,” Current Opinion in Plant Biology 13:160-166 (2010)). A genome sequence of a reference inbred line (B73) is currently available (Schnable et al., “The B73 Maize Genome: Complexity, Diversity, and Dynamics,” Science 326:1112-1115 (2009)). Most of the biomass in maize is contributed by the cell wall, which is composed of a network of cellulose, hemicelluloses, lignin, phenolic acid, lipids, and structural proteins (Penning et al., “Genetic Resources for Maize Cell Wall Biology,” Plant Physiol. 151:1703-1728 (2009)). In lignified tissue, lignin is heavily cross-linked with cellulose and hemicelluloses, thereby strengthening these tissues, and also increasing their resistance to digestion by ruminants as well as by bacteria and fungi (Sarkanen & Ludwig, Definition and Nomenclature. In Lignins: Occurrence, Formation, Structure and Reactions, Wiley-Interscience, New York, 1971). Because lignocellulosic biomass is a sustainable and renewable feedstock for agriculture and biofuels (Ragauskas et al., “The Path Forward for Biofuels and Biomaterials,” Science 311:484-489 (2006)), studies on the regulation of its composition have the potential to improve the quality of maize as a feed and for bio fuel production.

Brown midrib (bm) mutants in maize are characterized by the reddish-brown color of their leaf mid-ribs (Sattler et al., “Brown Midrib Mutations and their Importance to the Utilization of Maize, Sorghum, and Pearl Millet Lignocellulosic Tissues,” Plant Science 178:229-238 (2010)). The bm phenotype of maize was first reported over 80 years ago (Jorgenson, “Brown Midrib in Maize and its Linkage Relations,” Soc. Agron 549 (1931)), and it is now clear that the phenotype is associated with a reduction in lignin concentrations (Cherney et al., “Potential of Brown-midrib, Low-lignin Mutants for Improving Forage,” Adv. Agron. 46:157-198 (1991); Grand et al., “Comparison of Lignins and Enzymes Involved in Lignification in Normal and Brown Midrib (bm3) Mutant Corn Seedlings,” Physiol. Veg. 23:905-911 (1985); Sattler et al., “Brown Midrib Mutations and their Importance to the Utilization of Maize, Sorghum, and Pearl Millet Lignocellulosic Tissues,” Plant Science 178:229-238 (2010)). To date, six bm mutants (bm1 to bm6) have been identified. The bm1, bm2, bm3, bm4, bm5, and bm6 loci are located on chromosomes 5, 1, 4, 9, 5, and 2, respectively (Lawrence et al., “The Maize Genetics and Genomics Database. The Community Resource for Access to Diverse Maize Data,” Plant Physiol. 138:55-58 (2005); Sattler et al., “Brown Midrib Mutations and their Importance to the Utilization of Maize, Sorghum, and Pearl Millet Lignocellulosic Tissues,” Plant Science 178:229-238 (2010)). The bm3 and bm1 genes encode caffeic acid O-methyltransferase (COMT) (Vignols et al., “The Brown Midrib3 (bm3) Mutation in Maize Occurs in the Gene Encoding Caffeic Acid O-methyltransferase,” Plant Cell 7:407-416 (1995)) and cinnamyl alcohol dehydrogenase gene (CAD), respectively (Grand et al., “Comparison of Lignins and Enzymes Involved in Lignification in Normal and Brown Midrib (bm3) Mutant Corn Seedlings,” Physiol. Veg. 23:905-911 (1985)), both of which enzymes play key roles in lignin biosynthesis. The roles of other four bm genes in lignin biosynthesis are not clear. Therefore, cloning the remaining bm genes is expected to provide new insights into the regulation of lignin biosynthesis.

The present invention is directed to overcoming these and other deficiencies in the art.

### SUMMARY OF THE INVENTION

A first aspect of the present invention relates to a method for altering the concentration or composition of lignin in a plant. This method involves providing a transgenic plant or plant seed transformed with a nucleic acid construct effective in altering expression of an MTHFR protein capable of determining the concentration or composition of lignin in a plant and growing the transgenic plant or the plant grown from the transgenic plant seed under conditions effective to alter the concentration or composition of lignin in the transgenic plant or the plant grown from the transgenic plant seed.

A second aspect of the present invention relates to a plant produced by the method according to the first aspect of the present invention.

A third aspect of the present invention relates to a method for altering lignin concentration or composition in a plant. This method involves transforming a plant cell with a nucleic acid molecule encoding an MTHFR protein capable of altering lignin concentration or composition in a plant operably associated with a promoter to obtain a transformed plant cell. Expression of the nucleic acid molecule in the plant cell causes altered lignin concentration or composition relative to a nontransformed plant cell. The method further involves regenerating a plant from the transformed plant cell under conditions effective to alter lignin concentration or composition in the plant.

A fourth aspect of the present invention relates to a method of making a mutant plant having an altered level of MTHFR protein compared to that of a nonmutant plant. The mutant plant displays an altered lignin concentration or composition phenotype relative to a nonmutant plant. This method involves providing at least one cell of a nonmutant plant containing a gene encoding a functional MTHFR protein and treating the at least one cell of a nonmutant plant under conditions effective to inactivate or overactivate the gene, thereby yielding at least one mutant plant cell containing an inactivate or overactive MTHFR gene. The method further involves propagating the at least one mutant plant cell into a mutant plant. The mutant plant has an altered level of MTHFR protein compared to that of the nonmutant plant and displays an altered lignin concentration or composition phenotype relative to a nonmutant plant.

A fifth aspect of the present invention relates to a mutant plant produced according to the method according to the fourth aspect of the present invention.

A sixth aspect of the present invention relates to a mutant plant seed produced by growing the mutant plant according to the fifth aspect of the present invention under conditions effective to cause the mutant plant to produce seed.

A seventh aspect of the present invention relates to a method for altering lignin concentration or composition in a plant. This method involves transforming a plant cell with a nucleic acid molecule encoding a MTHFR protein capable of determining lignin concentration or composition in a plant operably associated with a promoter to obtain a transformed plant cell. A plant is regenerated from the transformed plant cell. The promoter is induced under conditions effective to alter lignin concentration or composition in the plant.

An eighth aspect of the present invention relates to a plant produced by the method according to the seventh aspect of the present invention.

A ninth aspect of the present invention relates to a method of identifying a candidate plant suitable for breeding that displays an altered lignin concentration or composition phenotype. This method involves analyzing the candidate plant for the presence, in its genome, of an inactive or overactive bm2 gene.

A tenth aspect of the present invention relates to a transgenic plant having an altered level of MTHFR protein capable of determining the lignin concentration or composition in a plant compared to that of a nontransgenic plant. The transgenic plant displays an altered lignin concentration or composition phenotype relative to a nontransgenic plant.

An eleventh aspect of the present invention relates to seed produced from the transgenic plant according to the tenth aspect of the present invention.

### BRIEF DESCRIPTION OF THE DRAWINGS

FIGS. 1A-C are photographs showing characterization of a bm2 mutant in maize. FIG. 1A shows the leaf structure of a greenhouse-grown bm2 mutant (bm2-ref) and a nonmutant wild-type (WT) maize. FIG. 1B shows adaxial and abaxial views of the midribs of bm2 mutant and WT maize. FIG. 1C shows histochemical staining of lignin of tissue sections from B73, bm2 mutant, and WT maize. Sections of midrib (a-c), stem (d-f), and root (g-i) were taken from B73 (a, d, g), bm2 mutant (b, e, h), and WT (c, f, i) maize that had been stained with phloroglucinol. Scale bar: 100 μm.

FIGS. 2A-C show MTHFR mRNA level in bm2 mutants. FIG. 2A shows RT-PCR gel analysis of the MTHFR mRNA levels in different tissues, including leaf, midrib, and root of wild-type (WT) and bm2-ref (bm2, Schnable Lab:Ac3247, 11B-418-1) plants. FIG. 2B shows RT-PCR gel analysis of MTHFR mRNA levels of bm2 mutants in four distinct genetic backgrounds: bm2 [1] (Schnable Lab:Ac3247, 10B-611-16); bm2 [2] (Schnable Lab:Ac3247, 11-1051); bm2 [3] (Schnable Lab:Ac3244, 11-1049), B73 (Ac660, 10B-613-9) and nonmutant (WT, Ac3247, 10-611-50) serve as control. FIG. 2C shows levels of MTHFR mRNA in midrib of wild-type and bm2-ref (Schnable Lab:Ac3247, 11-1051) with or without the Actinomycin D exposure (AMD, 50 g/ml, 12 hours). Incubation of the corresponding tissues in water and DMSO (AMD solvent) serve as mock experiments. Level of Actin, Cyclin (Cycl), or Gap mRNA serve as loading controls.

FIGS. 3A-D illustrate that bm2 encodes a putative MTHFR. FIG. 3A shows a representative phenotype of maize containing a Mu insertion in a putative MTHFR gene (bm2-Mu). FIG. 3B shows adaxial and abaxial views of midrib of nonmutant (WT) maize and bm2-Mu maize (bm2-Mu-11-2251). FIG. 3C shows gene structure of the MTHFR gene. The insertion sites of multiple Mu transposons are indicated by the open triangles in the first exon. FIG. 3D shows histochemical staining of lignin of tissue sections from midrib tissues from plants having the genotype bm2-Mu maize. Scale bar: 100 μm.

FIG. 4 shows the results of a yeast complementation assay. Growth of yeast strains are shown on Yeast-extract Peptone Dextrose (YPD, control), glucose SD-Met (control) plate, and galactose SD-Met (to induce expression of the insert at the plasmid) plate. MET11 (Mock): wild-type yeast transformed with plasmid without insert; met11 (Mock): MET11 knockout yeast transformed with plasmid without insert; met11 (Bm2): MET11 knockout yeast transformed with plasmid cloned with maize MTHFR; met11 (hMTHFR):MET11 knockout yeast transformed with plasmid cloned with human MTHFR (positive control of complementation assay). Their locations are labeled correspondingly at the circle.

FIG. 5 shows a comparison of RNA-Seq expression between bm2 and wild-type maize. Differences in gene expression patterns between the bm2-ref mutant (Schnable Lab: Ac3247, 10B-32) and wild-type (WT) controls were visualized using MapMan. Shaded boxes represent transcripts that are up-regulated and down-regulated in bm2 versus wild-type, respectively.

FIG. 6 is a schematic illustration showing identification of lignin biosynthetic genes that exhibit MTHFR-dependent and -independent patterns of transcript accumulation. Shaded boxes designate genes whose transcripts that are up-regulated and down-regulated in bm2 versus wild-type, respectively.

FIGS. 7A-D show identification of Mutator insertion alleles in the bm2 locus. FIG. 7A shows the gene structure of the MTHFR gene. The insertion sites of multiple Mu transposons are indicated by the open triangles in the first exon. The loci for annealing of primers for identifying Mutator insertion alleles in the bm2 are indicated. FIG. 7B shows a summary of the PCR results of the identification of Mutator insertion. FIG. 7C shows the primers used for the identification of Mutator insertion, including: bm2C1.1F_a (SEQ ID NO:1), bm2C1.1F_d (SEQ ID NO:2), bm2C1.1F_e (SEQ ID NO:3), bm2C1.3F_a (SEQ ID NO:4), bm2C1.4R_a (SEQ ID NO:5), and MuTIR (SEQ ID NO:6). FIG. 7D shows identification of Mutator insertion at MTHFR gene by PCR using primers bm2C1.1F_a (aligned on MTHFR) and MuTIR (aligned at Mu). Sequences of primers are stated at FIG. 7C. Genomic DNA of bm2 (Schnable Lab: Ac3247, 10B-611-16), B73 (Ac660, 10B-613-9) and an individual with the genetic background of Mutator without showing bm2 phenotype (WT, Mu background) serve as negative controls.

FIG. 8 is a chart showing that bm2 is located on chromosome 1 in maize. Each dot represents one of 46,289 SNP sites. The Y-axis indicates the probability of complete linkage between the SNP site and the mutant gene. The result is consistent with the reported position of the bm2 gene (Maize GDB).

FIGS. 9A-B show polymorphisms in the bm2-ref allele as compared to the Bm2-B73 wild-type allele. FIG. 9A shows the gene structure of the MTHFR gene. Black boxes indicate exons and lines between the boxes indicate introns. FIG. 9B shows the sequence alignment of the 3′ UTRs of the MTHFR gene of B73 (MTHFR, SEQ ID NO:7) and bm2 mutant (Schnable Lab: Ac3247, 10B-32) (bm2, SEQ ID NO:8). The 3′ UTR includes bases 1856 to 1373 of the mRNA. The stop codon at the MTHFR encoding sequence (TGA) is underlined. Point mutations, insertions, and deletions are shown. The Maize Genetics and Genomics Database (MGDB) accession number for MTHFR is 64885.

FIGS. 10A-F is a series of photographs showing histochemical staining of lignin of tissue sections from WT maize and bm2-Mu mutant. Sections of midrib (FIGS. 10A-B), stem (FIGS. 10C-D), and root (FIGS. 10E-F) were taken from WT (FIGS. 10A, 10C, 10E) and bm2-Mu mutant (FIGS. 10B, 10D, 10F) (bm2-Mu-10-7067E) that had been stained with phloroglucinol. Scale bar: 100 μm.

FIG. 11 shows the sequence alignment of deduced amino acid sequences of Maize Bm2 gene (SEQ ID NO:9) with the sequence of MET11 from Saccharomyces cerevisiae (SEQ ID NO:10).

FIG. 12 shows a summary of genotyping data of KASPar assay on chromosome 1 via fine mapping of the bm2 gene. In this mapping population, 537 plants germinated. 41 recombinants (dotted box) were identified by using flanking markers within 2 MB intervals, using primers bm2-289632923 and bm2-291983683. Six recombinants (dashed box) were found by using flanking makers within 0.51 MB intervals, using primers bm2-290599983 and bm2-291111263. The light highlight indicates homozygote bm2 allele. Three with dark highlight indicate heterozygote bm2 allele. Zero indicates no data. Chromosome 1 model is at the lower panel of the genotyping summary table to visualize the relative distance between the flanking markers.

### DETAILED DESCRIPTION OF THE INVENTION

One aspect of the present invention relates to a method for altering the concentration or composition of lignin in a plant. This method involves providing a transgenic plant or plant seed transformed with a nucleic acid construct effective in altering expression of an MTHFR protein capable of determining the concentration or composition of lignin in a plant and growing the transgenic plant or the plant grown from the transgenic plant seed under conditions effective to alter the concentration or composition of lignin in the transgenic plant or the plant grown from the transgenic plant seed.

Plants include any plant with a bm2 gene, including monocots and dicots, crop plants and ornamental plants. For example, plants include, without limitation, maize, sorghum, sudangrass, pearl millet, alfalfa, rice, wheat, barley, rye, cotton, sunflower, peanut, corn, potato, sweet potato, kidney bean, pea, chicory, lettuce, endive, cabbage, bok Choy, brussel sprout, beet, parsnip, turnip, cauliflower, broccoli, radish, spinach, onion, garlic eggplant, pepper, celery, carrot, squash, pumpkin, zucchini, cucumber, apple, pear, melon, citrus, peach, strawberry, grape, raspberry, pineapple, soybean, Medicago, tobacco, tomato, sugarcane, Arabidopsis thaliana, Saintpauliai, Populus, Miscanthus, switchgrass, conifer trees, deciduous trees, forage pasture and hay crops, petunia, pelargonium, poinsettia, chrysanthemum, carnation, zinnia, turfgrass, lily, and nightshade.

As used herein, altering expression of an MTHFR protein is carried out via well-known methods in the art for up-regulation, down-regulation, ectopic expression, or gene silencing. By gene silencing, it is meant the interruption or suppression of the expression of a gene at the level of transcription or translation. Other means of altering protein expression are being developed. For example, epigenetics is the study of heritable changes in gene expression or cellular phenotype caused by mechanisms other than changes in the underlying DNA sequence. Epigenetics refers to functionally relevant modifications to the genome that do not involve a change in the nucleotide sequence. Examples of such changes are DNA methylation and histone deacetylation, both of which serve to suppress gene expression without altering the sequence of the silenced genes.

In one embodiment, the above step of providing includes providing a nucleic acid construct having a nucleic acid molecule configured to silence or enhance MTHFR protein expression. The construct also includes a 5′ DNA promoter sequence and a 3′ terminator sequence. The nucleic acid molecule, the promoter, and the terminator are operatively coupled to permit expression of the nucleic acid molecule. A plant cell is then transformed with the nucleic acid construct. The method can further involve propagating plants from the transformed plant cell. Suitable methods for transforming the plant can include, for example, Agrobacterium-mediated transformation, vacuum infiltration, biolistic transformation, electroporation, micro-injection, chemical-mediated transformation (e.g., polyethylene-mediated transformation), and/or laser-beam transformation. The various aspects of this method are described in more detail infra.

In one embodiment, the nucleic acid construct is configured to enhance MTHFR protein expression. Over-expression of a protein can be carried out by methods well-known in the art, including using a transcription factor or by ectopic expression.

In another embodiment, the nucleic acid construct results in suppression or interruption or interference of MTHFR protein expression. Silencing of MTHFR protein expression may be carried out by the nucleic acid molecule of the construct containing a dominant negative mutation and encoding a non-functional MTHFR protein.

In another embodiment, the nucleic acid construct results in interference of MTHFR protein expression by sense or co-suppression in which the nucleic acid molecule of the construct is in a sense (5′→3′) orientation. Co-suppression has been observed and reported in many plant species and may be subject to a transgene dosage effect or, in another model, an interaction of endogenous and transgene transcripts that results in aberrant mRNAs (Senior, “Uses of Plant Gene Silencing,” Biotechnology and Genetic Engineering Reviews 15:79-119 (1998); Waterhouse et al., “Exploring Plant Genomes by RNA-Induced Gene Silencing,” Nature Review: Genetics 4:29-38 (2003), which are hereby incorporated by reference in their entirety). A construct with the nucleic acid molecule in the sense orientation may also give sequence specificity to RNA silencing when inserted into a vector along with a construct of both sense and antisense nucleic acid orientations as described infra (Wesley et al., “Construct Design for Efficient, Effective and High-Throughput Gene Silencing in Plants,” Plant Journal 27(6):581-590 (2001), which is hereby incorporated by reference in its entirety).

In yet another embodiment, the nucleic acid construct results in interference of MTHFR protein expression by the use of antisense suppression in which the nucleic acid molecule of the construct is an antisense (3′→5′) orientation. The use of antisense RNA to down-regulate the expression of specific plant genes is well known (van der Krol et al., “An Anti-sense Chalcone Synthase Gene in Transgenic Plants Inhibits Flower Pigmentation,” Nature 333:866-869 (1988) and Smith et al., “Antisense RNA Inhibition of Polygalacturonase Gene Expression in Transgenic Tomatoes,” Nature 334:724-726 (1988), which are hereby incorporated by reference in their entirety). Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). In the target cell, the antisense nucleic acids hybridize to a target nucleic acid and interfere with transcription, and/or RNA processing, transport, translation, and/or stability. The overall effect of such interference with the target nucleic acid function is the disruption of protein expression (Baulcombe, “Mechanisms of Pathogen-Derived Resistance to Viruses in Transgenic Plants,” Plant Cell 8:1833-44 (1996); Dougherty, et al., “Transgenes and Gene Suppression Telling us Something New?,” Current Opinion in Cell Biology 7:399-05 (1995); Lomonossoff, “Pathogen-Derived Resistance to Plant Viruses,” Ann. Rev. Phytopathol. 33:323-43 (1995), which are hereby incorporated by reference in their entirety). Accordingly, one embodiment involves a nucleic acid construct which contains the MTHFR protein encoding nucleic acid molecule being inserted into the construct in antisense orientation.

Interference of MTHFR protein expression is also achieved in the present invention by the generation of double-stranded RNA (“dsRNA”) through the use of inverted-repeats, segments of gene-specific sequences oriented in both sense and antisense orientations. In one embodiment, sequences in the sense and antisense orientations are linked by a third segment, and inserted into a suitable expression vector having the appropriate 5′ and 3′ regulatory nucleotide sequences operably linked for transcription. The expression vector having the modified nucleic acid molecule is then inserted into a suitable host cell or subject. In the present invention, the third segment linking the two segments of sense and antisense orientation may be any nucleotide sequence such as a fragment of the β-glucuronidase (“GUS”) gene. In another embodiment, a functional (splicing) intron of the MTHFR gene may be used for the third (linking) segment, or, in yet another aspect of the present invention, other nucleotide sequences without complementary components in the MTHFR gene may be used to link the two segments of sense and antisense orientation (Chuang et al., “Specific and Heritable Genetic Interference by Double-Stranded RNA in Arabidopsis thaliana,” Proc. Nat'l. Academy of Sciences USA 97(9):4985-4990 (2000); Smith et al., “Total Silencing by Intron-Spliced Hairpin RNAs,” Nature 407:319-320 (2000); Waterhouse et al., “Exploring Plant Genomes by RNA-Induced Gene Silencing,” Nature Review: Genetics 4:29-38 (2003); Wesley et al., “Construct Design for Efficient, Effective and High-Throughput Gene Silencing in Plants,” Plant Journal 27(6):581-590 (2001), which are hereby incorporated by reference in their entirety). In any of the embodiments with inverted repeats of MTHFR protein, the sense and antisense segments may be oriented either head-to-head or tail-to-tail in the construct.

Another embodiment involves using hairpin RNA (“hpRNA”) which may also be characterized as dsRNA. This involves RNA hybridizing with itself to form a hairpin structure that comprises a single-stranded loop region and a base-paired stem. Though a linker may be used between the inverted repeat segments of sense and antisense sequences to generate hairpin or double-stranded RNA, the use of intron-free hpRNA can also be used to achieve silencing of MTHFR protein expression.

Alternatively, in another embodiment, a plant may be transformed with constructs encoding both sense and antisense orientation molecules having separate promoters and no third segment linking the sense and antisense sequences (Chuang et al., “Specific and Heritable Genetic Interference by Double-Stranded RNA in Arabidopsis thaliana,” Proc. Nat'l. Academy of Sciences USA 97(9):4985-4990 (2000); Waterhouse et al., “Exploring Plant Genomes by RNA-Induced Gene Silencing,” Nature Review: Genetics 4:29-38 (2003); Wesley et al., “Construct Design for Efficient, Effective and High-Throughput Gene Silencing in Plants,” Plant Journal 27(6):581-590 (2001), which are hereby incorporated by reference in their entirety).

The nucleotide sequences used in the present invention may be inserted into any of the many available expression vectors and cell systems using reagents that are well known in the art. Suitable vectors include, but are not limited to, the following viral vectors such as lambda vector system gt11, gt WES.tB, Charon 4, and plasmid vectors such as pG-Cha, p35S-Cha, pBR322, pBR325, pACYC177, pACYC1084, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see Studier et al., “Use of T7 RNA Polymerase to Direct Expression of Cloned Genes,” Gene Expression Technology vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Spring Harbor, N.Y.: Cold Spring Harbor Press (1989), and Ausubel et al., Current Protocols in Molecular Biology, New York, N.Y.: John Wiley & Sons (1989), which are hereby incorporated by reference in their entirety.

In preparing a nucleic acid construct for expression, the various nucleic acid sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique and target species for transformation. A variety of vectors are available for stable transformation using Agrobacterium tumefaciens, a soilborne bacterium that causes crown gall. Crown gall is characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTi, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens (Fraley et al., “Expression of Bacterial Genes in Plant Cells,” Proc. Nat'l Acad. Sci. 80:4803-4807 (1983), which is hereby incorporated by reference in its entirety).

Further improvement of this technique led to the development of the binary vector system (Bevan, “Binary Agrobacterium Vectors for Plant Transformation,” Nucleic Acids Res. 12:8711-8721 (1984), which is hereby incorporated by reference in its entirety). In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens, and contains a multiclonal site that facilitates the cloning of a transgene. An example of a commonly-used vector is pBin19 (Frisch et al., “Complete Sequence of the Binary Vector Bin19,” Plant Mol. Biol. 27:405-409 (1995), which is hereby incorporated by reference in its entirety). Any appropriate vectors now known or later described for genetic transformation are suitable for use with the present invention.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Certain “control elements” or “regulatory sequences” are also incorporated into the vector-construct. These include non-translated regions of the vector, promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used. Tissue-specific and organ-specific promoters can also be used.

A constitutive promoter is a promoter that directs expression of a gene throughout the development and life of an organism. Examples of some constitutive promoters that are widely used for inducing expression of transgenes include the nopaline synthase (NOS) gene promoter, from Agrobacterium tumefaciens (U.S. Pat. No. 5,034,322 to Rogers et al., which is hereby incorporated by reference in its entirety), the cauliflower mosaic virus (CaMV) 35S and 19S promoters (U.S. Pat. No. 5,352,605 to Fraley et al., which is hereby incorporated by reference in its entirety), those derived from any of the several actin genes, which are known to be expressed in most cells types (U.S. Pat. No. 6,002,068 to Privalle et al., which is hereby incorporated by reference in its entirety), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide, or phenolic compound, or a physiological stress directly imposed upon the plant such as cold, heat, salt, toxins, or through the action of a pathogen or disease agent such as a virus or fungus. A plant cell containing an inducible promoter may be exposed to an inducer by externally applying the inducer to the cell or plant such as by spraying, watering, heating, or by exposure to the operative pathogen. An example of an appropriate inducible promoter is a glucocorticoid-inducible promoter (Schena et al., “A Steroid-Inducible Gene Expression System for Plant Cells,” Proc. Natl. Acad. Sci. 88:10421-5 (1991), which is hereby incorporated by reference in its entirety). Expression of the transgene-encoded protein is induced in the transformed plants when the transgenic plants are brought into contact with nanomolar concentrations of a glucocorticoid, or by contact with dexamethasone, a glucocorticoid analog (Schena et al., “A Steroid-Inducible Gene Expression System for Plant Cells,” Proc. Natl. Acad. Sci. USA 88:10421-5 (1991); Aoyama et al., “A Glucocorticoid-Mediated Transcriptional Induction System in Transgenic Plants,” Plant J. 11:605-612 (1997); McNellis et al., “Glucocorticoid-Inducible Expression of a Bacterial Avirulence Gene in Transgenic Arabidopsis Induces Hypersensitive Cell Death, Plant J. 14(2):247-57 (1998), which are hereby incorporated by reference in their entirety). In addition, inducible promoters include promoters that function in a tissue specific manner to regulate the gene of interest within selected tissues of the plant. Examples of such tissue specific or developmentally regulated promoters include seed, flower, fruit, or root specific promoters as are well known in the field (U.S. Pat. No. 5,750,385 to Shewmaker et al., which is hereby incorporated by reference in its entirety).

A number of tissue- and organ-specific promoters have been developed for use in genetic engineering of plants (Potenza et al., “Targeting Transgene Expression in Research, Agricultural, and Environmental Applications: Promoters used in Plant Transformation,” In Vitro Cell. Dev. Biol. Plant 40:1-22 (2004), which is hereby incorporated by reference in its entirety). Examples of such promoters include those that are floral-specific (Annadana et al., “Cloning of the Chrysanthemum UEP1 Promoter and Comparative Expression in Florets and Leaves of Dendranthema grandiflora,” Transgenic Res. 11:437-445 (2002), which is hereby incorporated by reference in its entirety), seed-specific (Kluth et al., “5′ Deletion of a gbss1 Promoter Region Leads to Changes in Tissue and Developmental Specificities,” Plant Mol. Biol. 49:669-682 (2002), which is hereby incorporated by reference in its entirety), root-specific (Yamamoto et al., “Characterization of cis-acting Sequences Regulating Root-Specific Gene Expression in Tobacco,” Plant Cell 3:371-382 (1991), which is hereby incorporated by reference in its entirety), fruit-specific (Fraser et al., “Evaluation of Transgenic Tomato Plants Expressing an Additional Phytoene Synthase in a Fruit-Specific Manner,” Proc. Natl. Acad. Sci. USA 99:1092-1097 (2002), which is hereby incorporated by reference in its entirety), and tuber/storage organ-specific (Visser et al., “Expression of a Chimaeric Granule-Bound Starch Synthase-GUS gene in transgenic Potato Plants,” Plant Mol. Biol. 17:691-699 (1991), which is hereby incorporated by reference in its entirety). Targeted expression of an introduced gene (transgene) is necessary when expression of the transgene could have detrimental effects if expressed throughout the plant. On the other hand, silencing a gene throughout a plant could also have negative effects. However, this problem could be avoided by localizing the silencing to a region by a tissue-specific promoter.

The nucleic acid construct also includes an operable 3′ regulatory region, selected from among those which are capable of providing correct transcription termination and polyadenylation of mRNA for expression in the host cell of choice, operably linked to a modified trait nucleic acid molecule of the present invention. A number of 3′ regulatory regions are known to be operable in plants. Exemplary 3′ regulatory regions include, without limitation, the nopaline synthase (“nos”) 3′ regulatory region (Fraley et al., “Expression of Bacterial Genes in Plant Cells,” Proc. Nat'l Acad. Sci. USA 80:4803-4807 (1983), which is hereby incorporated by reference in its entirety) and the cauliflower mosaic virus (“CaMV”) 3′ regulatory region (Odell et al., “Identification of DNA Sequences Required for Activity of the Cauliflower Mosaic Virus ³⁵S Promoter,” Nature 313(6005):810-812 (1985), which is hereby incorporated by reference in its entirety). Virtually any 3′ regulatory region known to be operable in plants would be suitable for use in conjunction with the present invention.

The different components described above can be ligated together to produce the expression systems which contain the nucleic acid constructs used in the present invention, using well known molecular cloning techniques as described in Sambrook et al., Molecular Cloning: A Laboratory Manual, Second Edition Cold Spring Harbor, N.Y.: Cold Spring Harbor Press (1989), and Ausubel et al. Current Protocols in Molecular Biology, New York, N.Y.: John Wiley & Sons (1989), which are hereby incorporated by reference in their entirety.

Once the nucleic acid construct has been prepared, it is ready to be incorporated into a host cell. Basically, this method is carried out by transforming a host cell with the nucleic acid construct under conditions effective to achieve transcription of the nucleic acid molecule in the host cell. This is achieved with standard cloning procedures known in the art, such as described by Sambrook et al., Molecular Cloning: A Laboratory Manual, Second Edition, Cold Spring Harbor Laboratory, Cold Spring Harbor, N.Y. (1989), which is hereby incorporated by reference in its entirety. Suitable host cells are plant cells. Methods of transformation may result in transient or stable expression of the nucleic acid under control of the promoter. Preferably, the nucleic acid construct of the present invention is stably inserted into the genome of the recombinant plant cell as a result of the transformation, although transient expression can serve an important purpose, particularly when the plant under investigation is slow-growing.

Plant tissue suitable for transformation includes leaf tissue, root tissue, meristems, zygotic and somatic embryos, callus, protoplasts, tassels, pollen, embryos, anthers, and the like. The means of transformation chosen is that most suited to the tissue to be transformed.

Transient expression in plant tissue can be achieved by particle bombardment (Klein et al., “High-Velocity Microprojectiles for Delivering Nucleic Acids Into Living Cells,” Nature 327:70-73 (1987), which is hereby incorporated by reference in its entirety), also known as biolistic transformation of the host cell, as discussed in U.S. Pat. Nos. 4,945,050, 5,036,006, and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety.

In particle bombardment, tungsten or gold microparticles (1 to 2 μm in diameter) are coated with the DNA of interest and then bombarded at the tissue using high pressure gas. In this way, it is possible to deliver foreign DNA into the nucleus and obtain a temporal expression of the gene under the current conditions of the tissue. Biologically active particles (e.g., dried bacterial cells containing the vector and heterologous DNA) can also be propelled into plant cells. Other variations of particle bombardment, now known or hereafter developed, can also be used.

An appropriate method of stably introducing the nucleic acid construct into plant cells is to infect a plant cell with Agrobacterium tumefaciens or Agrobacterium rhizogenes previously transformed with the nucleic acid construct. As described above, the Ti (or RI) plasmid of Agrobacterium enables the highly successful transfer of a foreign nucleic acid molecule into plant cells. A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety).

Yet another method of introduction is fusion of protoplasts with other entities, either minicells, cells, lysosomes, or other fusible lipid-surfaced bodies (Fraley et al., “Liposome-mediated Delivery of Tobacco Mosaic Virus RNA into Tobacco Protoplasts: A Sensitive Assay for Monitoring Liposome-protoplast Interactions,” Proc. Natl. Acad. Sci. USA 79:1859-63 (1982), which is hereby incorporated by reference in its entirety). The nucleic acid molecule may also be introduced into the plant cells by electroporation (Fromm et al., “Expression of Genes Transferred into Monocot and Dicot Plant Cells by Electroporation,” Proc. Natl. Acad. Sci. USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate. Other methods of transformation include chemical-mediated plant transformation, micro-injection, physical abrasives, and laser beams (Senior, “Uses of Plant Gene Silencing,” Biotechnology and Genetic Engineering Reviews 15:79-119 (1998), which is hereby incorporated by reference in its entirety). The precise method of transformation is not critical to the practice of the present invention. Any method that results in efficient transformation of the host cell of choice is appropriate for practicing the present invention.

After transformation, the transformed plant cells must be regenerated. Plant regeneration from cultured protoplasts is described in Evans et al., Handbook of Plant Cell Cultures, Vol. 1, New York, N.Y.: MacMillan Publishing Co. (1983); Vasil, ed., Cell Culture and Somatic Cell Genetics of Plants, Vol. I (1984) and Vol. III (1986), Orlando: Acad. Press; and Fitch et al., “Somatic Embryogenesis and Plant Regeneration from Immature Zygotic Embryos of Papaya (Carica papaya L.),” Plant Cell Rep. 9:320 (1990), which are hereby incorporated by reference in their entirety.

Means for regeneration vary from species to species of plants, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins Efficient regeneration will depend on the medium, on the genotype, and on the history of the culture. If these three variables are controlled, then regeneration is usually reproducible and repeatable.

Preferably, transformed cells are first identified using a selection marker simultaneously introduced into the host cells along with the nucleic acid construct of the present invention. Suitable selection markers include, without limitation, markers encoding for antibiotic resistance, such as the neomycin phosphotransferae II (“nptII”) gene which confers kanamycin resistance (Fraley et al., “Expression of Bacterial Genes in Plant Cells,” Proc. Natl. Acad. Sci. USA 80:4803-4807 (1983), which is hereby incorporated by reference in its entirety), and the genes which confer resistance to gentamycin, G418, hygromycin, streptomycin, spectinomycin, tetracycline, chloramphenicol, and the like. Cells or tissues are grown on a selection medium containing the appropriate antibiotic, whereby generally only those transformants expressing the antibiotic resistance marker continue to grow. Other types of markers are also suitable for inclusion in the expression cassette of the present invention. For example, a gene encoding for herbicide tolerance, such as tolerance to sulfonylurea is useful, or the dhfr gene, which confers resistance to methotrexate (Bourouis et al., “Vectors Containing a Prokaryotic Dihydrofolate Reductase Gene Transform Drosophila Cells to Methotrexate-resistance,” EMBO J. 2:1099-1104 (1983), which is hereby incorporated by reference in its entirety). Similarly, “reporter genes,” which encode for enzymes providing for production of an identifiable compound are suitable. The most widely used reporter gene for gene fusion experiments has been uidA, a gene from Escherichia coli that encodes the β-glucuronidase protein, also known as GUS (Jefferson et al., “GUS Fusions: β Glucuronidase as a Sensitive and Versatile Gene Fusion Marker in Higher Plants,” EMBO J. 6:3901-3907 (1987), which is hereby incorporated by reference in its entirety). Similarly, enzymes providing for production of a compound identifiable by luminescence, such as luciferase, are useful. The selection marker employed will depend on the target species; for certain target species, different antibiotics, herbicide, or biosynthesis selection markers are preferred.

Plant cells and tissues selected by means of an inhibitory agent or other selection marker are then tested for the acquisition of the transgene (Sambrook et al., Molecular Cloning: A Laboratory Manual, Cold Spring Harbor, N.Y.: Cold Spring Harbor Press (1989), which is hereby incorporated by reference in its entirety).

After the fusion gene containing a nucleic acid construct is stably incorporated in transgenic plants, the transgene can be transferred to other plants by sexual crossing. Any of a number of standard breeding techniques can be used, depending upon the species to be crossed. Once transgenic plants of this type are produced, the plants themselves can be cultivated in accordance with conventional procedure so that the nucleic acid construct is present in the resulting plants. Alternatively, transgenic seeds are recovered from the transgenic plants. These seeds can then be planted in the soil and cultivated using conventional procedures to produce transgenic plants.

An example of an MTHFR protein encoded by the nucleic acid molecule used in the present invention is an MTHFR protein from maize having an amino acid sequence of SEQ ID NO:11 as follows:

MKVIEKILEA AGDGRTAFSF EYFPPKTEEG VENLFERMDR
MVAHGPSFCD ITWGAGGSTA DLTLEIANRM QNMVCVETMM
HLTCTNMPVE KIDHALETIK SNGIQNVLAL RGDPPHGQDK
FVQVEGGFAC ALDLVQHIRA KYGDYFGITV AGYPEAHPDA
IQGEGGATLE AYSNDLAYLK RKVDAGADLI VTQLFYDTDI
FLKFVNDCRQ IGITCPIVPG IMPINNYKGF LRMTGFCKTK
IPSEITAALD PIKDNEEAVR QYGIHLGTEM CKKILATGIK
TLHLYTLNMD KSAIGILMNL GLIEESKVSR PLPWRPATNV
FRVKEDVRPI FWANRPKSYL KRTLGWDQYP HGRWGDSRNP
SYGALTDHQF TRPRGRGKKL QEEWAVPLKS VEDISERFTN
FCQGKLTSSP WSELDGLQPE TKIIDDQLVN INQKGFLTIN
SQPAVNGEKS DSPTVGWGGP GGYVYQKAYL EFFCAKEKLD
QLIEKIKAFP SLTYIAVNKD GETFSNISPN AVNAVTWGVF
PGKEIIQPTV VDHASFMVWK DEAFEIWTRG WGCMFPEGDS
SRELLEKVQK TYYLVSLVDN DYVQGDLFAA FKI

Other examples of MTHFR proteins from various other species are set forth in Table 4.

TABLE 4
MTHFR Protein Sequences

| | NCBI Ref. | | NCBI Ref. |
|---|---|---|---|
| Species | Seq. ID | Species | Seq. ID |
| Saccharomyces | NP_015302.1 | Candida dubliniensis | XP_002419224.1 |
| cerevisiae | | | |
| Arabidopsis thaliana | NP_191556.1 | Sorghum bicolor | XP_002463656.1 |
| Arabidopsis thaliana | NP_566011.1 | Talaromyces | XP_002481086.1 |
| | | stipitatus | |
| Schizosaccharomyces | NP_593224.1 | Talaromyces | XP_002481889.1 |
| pombe | | stipitatus | |
| Caenorhabditis | NP_741027.1 | Komagataella | XP_002492060.1 |
| elegans | | pastoris | |
| Caenorhabditis | NP_741028.1 | Zygosaccharomyces | XP_002499028.1 |
| elegans | | rouxii | |
| Saccharomyces | NP_011390.2 | Ricinus communis | XP_002529223.1 |
| cerevisiae S288c | | | |
| Arabidopsis thaliana | NP_850723.1 | Lachancea | XP_002553955.1 |
| | | thermotolerans | |
| Arabidopsis thaliana | NP_850724.1 | Lachancea | XP_002556159.1 |
| | | thermotolerans | |
| Mus musculus | NP_034970.2 | Candida tropicalis | XP_002548276.1 |
| Gibberella zeae PH-1 | XP_387303.1 | Penicillium | XP_002560275.1 |
| | | chrysogenum | |
| Gibberella zeae PH-1 | XP_389748.1 | Uncinocarpus reesii | XP_002544363.1 |
| Candida glabrata | XP_446274.1 | Uncinocarpus reesii | XP_002542937.1 |
| CBS 138 | | | |
| Kluyveromyces lactis | XP_453605.1 | Branchiostoma | XP_002613844.1 |
| NRRL Y-1140 | | floridae | |
| Kluyveromyces lactis | XP_455518.1 | Clavispora | XP_002616943.1 |
| NRRL Y-1140 | | lusitaniae | |
| Yarrowia lipolytica | XP_500349.1 | Ajellomyces | XP_002629356.1 |
| | | dermatitidis | |
| Methylococcus | YP_112676.1 | Caenorhabditis | XP_002630522.1 |
| capsulatus str. Bath | | briggsae | |
| Cryptococcus | XP_568023.1 | Pirellula staleyi | YP_003370301.1 |
| neoformans var. | | | |
| neoformans JEC21 | | | |
| Bos taurus | NP_001011685.1 | Conexibacter woesei | YP_003392888.1 |
| Dictyostelium | XP_641844.1 | Naegleria gruberi | XP_002681849.1 |
| discoideum AX4 | | | |
| Aspergillus nidulans | XP_663487.1 | Saccoglossus | XP_002737338.1 |
| FGSC A4 | | kowalevskii | |
| Aspergillus nidulans | XP_681484.1 | Oryctolagus | XP_002721570.1 |
| | | cuniculus | |
| Candida albicans | XP_720117.1 | Rattus norvegicus | XP_001074061.2 |
| Candida albicans | XP_717124.1 | Debaryomyces | XP_002770555.1 |
| | | hansenii | |
| Aspergillus fumigatus | XP_747996.1 | Perkinsus marinus | XP_002776404.1 |
| Aspergillus fumigatus | XP_755463.1 | Perkinsus marinus | XP_002788701.1 |
| Ustilago maydis | XP_760759.1 | Paracoccidioides | XP_002794022.1 |
| | | brasiliensis | |
| Strongylocentrotus | XP_794308.1 | Paracoccidioides | XP_002794072.1 |
| purpuratus | | brasiliensis | |
| Thiobacillus | YP_316275.1 | Callithrix jacchus | XP_002750351.1 |
| denitrificans | | | |
| Neurospora crassa | XP_961729.1 | Tuber melanosporum | XP_002841580.1 |
| Homo sapiens | NP_005948.3 | Arthroderma otae | XP_002849288.1 |
| Macaca mulatta | XP_001105017.1 | Meiothermus | YP_003684099.1 |
| | | silvanus | |
| Pan troglodytes | XP_001141040.1 | Pongo abelii | XP_002811545.1 |
| Mariprofundus | ZP_01453482.1 | Arabidopsis lyrata | XP_002876531.1 |
| ferrooxydans | | subsp. lyrata | |
| Aspergillus terreus | XP_001208812.1 | Arabidopsis lyrata | XP_002880088.1 |
| | | subsp. lyrata | |
| Oryza sativa | NP_001051683.1 | Arabidopsis lyrata | XP_002880090.1 |
| | | subsp. lyrata | |
| Chaetomium globosum | XP_001223823.1 | Coprinopsis cinerea | XP_001831373.2 |
| Coccidioides immitis | XP_001241039.1 | Phytophthora | XP_002999032.1 |
| | | infestans | |
| Coccidioides immitis | XP_001242664.1 | Phytophthora | XP_002909846.1 |
| | | infestans | |
| Neosartorya fischeri | XP_001260599.1 | Ailuropoda | XP_002922800.1 |
| | | melanoleuca | |
| Neosartorya fischeri | XP_001266187.1 | Ashbya gossypii | NP_982713.2 |
| Aspergillus clavatus | XP_001275420.1 | Verticillium albo- | XP_003008442.1 |
| | | atrum | |
| Aspergillus niger | XP_001399463.1 | Verticillium albo- | XP_003009170.1 |
| | | atrum | |
| Magnaporthe oryzae | XP_362588.2 | Arthroderma | XP_003012369.1 |
| | | benhamiae | |
| Magnaporthe oryzae | XP_363802.2 | Arthroderma | XP_003015086.1 |
| | | benhamiae | |
| Leishmania infantum | XP_001469364.1 | Trichophyton | XP_003018616.1 |
| | | verrucosum | |
| Meyerozyma | XP_001482693.1 | Trichophyton | XP_003020646.1 |
| guilliermondii | | verrucosum | |
| Xenopus laevis | NP_001080092.1 | Schizophyllum | XP_003032824.1 |
| | | commune | |
| Lodderomyces | XP_001527283.1 | Selaginella | XP_002960599.1 |
| elongisporus | | moellendorffii | |
| Equus caballus | XP_001492020.1 | Selaginella | XP_002969241.1 |
| | | moellendorffii | |
| Scheffersomyces | XP_001384247.2 | Selaginella | XP_002979895.1 |
| stipitis | | moellendorffii | |
| Ajellomyces capsulatus | XP_001537685.1 | Selaginella | XP_002987373.1 |
| | | moellendorffii | |
| Botryotinia fuckeliana | XP_001547755.1 | Volvox carteri | XP_002954594.1 |
| | | f. nagariensis | |
| Botryotinia fuckeliana | XP_001558649.1 | Nectria | XP_003046086.1 |
| | | haematococca | |
| Leishmania | XP_001569284.1 | Nectria | XP_003050376.1 |
| braziliensis | | haematococca | |
| Sclerotinia | XP_001589131.1 | Coccidioides | XP_003065064.1 |
| sclerotiorum | | posadasii | |
| Sclerotinia | XP_001597813.1 | Coccidioides | XP_003069839.1 |
| sclerotiorum | | posadasii | |
| Nematostella vectensis | XP_001632898.1 | Caenorhabditis | XP_003102472.1 |
| | | remanei | |
| Nematostella vectensis | XP_001633891.1 | Stigmatella | YP_003954649.1 |
| | | aurantiaca | |
| Xenopus (Silurana) | NP_001096464.1 | Sus scrofa | XP_003127623.1 |
| tropicalis | | | |
| Leishmania major | XP_001687229.1 | Sus scrofa | XP_003127622.1 |
| strain | | | |
| Chlamydomonas | XP_001702476.1 | Loa loa | XP_003144175.1 |
| reinhardtii | | | |
| Zea mays | NP_001104947.1 | Arthroderma | XP_003174054.1 |
| | | gypseum | |
| Malassezia globosa | XP_001729491.1 | Arthroderma | XP_003175411.1 |
| | | gypseum | |
| Monosiga brevicollis | XP_001746001.1 | Aspergillus oryzae | XP_001822709.2 |
| Physcomitrella patens | XP_001758327.1 | Cryptococcus gattii | XP_003193407.1 |
| Physcomitrella patens | XP_001785862.1 | Meleagris gallopavo | XP_003212375.1 |
| Phaeosphaeria | XP_001801240.1 | Trichophyton | XP_003234157.1 |
| nodorum | | rubrum | |
| Phaeosphaeria | XP_001802911.1 | Trichophyton | XP_003235502.1 |
| nodorum | | rubrum | |
| Aspergillus oryzae | XP_001821959.1 | Dictyostelium | XP_003288595.1 |
| | | purpureum | |
| Laccaria bicolor | XP_001875153.1 | Pyrenophora teres | XP_003298697.1 |
| Brugia malayi | XP_001898544.1 | Puccinia graminis | XP_003320871.1 |
| | | f. sp. tritici | |
| Podospora anserina | XP_001910226.1 | Monodelphis | XP_001371469.2 |
| | | domestica | |
| Pyrenophora tritici- | XP_001941642.1 | Sordaria | XP_003349970.1 |
| repentis | | macrospora | |
| Danio rerio | NP_001121727.1 | Amphimedon | XP_003387898.1 |
| | | queenslandica | |
| Trichoplax adhaerens | XP_002113874.1 | Loxodonta africana | XP_003413212.1 |
| Chthoniobacter flavus | ZP_03129291.1 | Ornithorhynchus | XP_001516302.2 |
| | | anatinus | |
| Ciona intestinalis | XP_002131246.1 | Canis lupus | XP_535405.3 |
| | | familiaris | |
| Penicillium marneffei | XP_002147724.1 | Oreochromis | XP_003454316.1 |
| | | niloticus | |
| Penicillium marneffei | XP_002152051.1 | Cavia porcellus | XP_003471471.1 |
| Schizosaccharomyces | XP_002174847.1 | Cricetulus griseus | XP_003514213.1 |
| japonicus | | | |
| Phaeodactylum | XP_002181165.1 | Glycine max | XP_003523478.1 |
| tricornutum | | | |
| Phaeodactylum | XP_002184308.1 | Glycine max | XP_003527588.1 |
| tricornutum | | | |
| Thioalkalivibrio | YP_002514956.1 | Brachypodium | XP_003563842.1 |
| sulfidophilus | | distachyon | |
| Hydra magnipapillata | XP_002159652.1 | Medicago truncatula | XP_003589823.1 |
| Thalassiosira | XP_002286373.1 | Vitis vinifera | XP_003632105.1 |
| pseudonana | | | |
| Thalassiosira | XP_002288069.1 | Gallus gallus | XP_417645.3 |
| pseudonana | | | |
| Taeniopygia guttata | XP_002193682.1 | Eremothecium | XP_003645392.1 |
| | | cymbalariae | |
| Populus trichocarpa | XP_002310366.1 | Naumovozyma | XP_003670383.1 |
| | | dairenensis | |
| Populus trichocarpa | XP_002331635.1 | Naumovozyma | XP_003671989.1 |
| | | dairenensis | |
| Taeniopygia guttata | XP_002194351.1 | Naumovozyma | XP_003675609.1 |
| | | castellii | |
| Vitis vinifera | XP_002272730.1 | Naumovozyma | XP_003676256.1 |
| | | castellii | |
| Aspergillus flavus | XP_002382828.1 | Tetrapisispora | XP_003687438.1 |
| | | phaffii | |
| Mus musculus | NP_001155270.1 | Torulaspora | XP_003682926.1 |
| | | delbrueckii | |
| Candida dubliniensis | XP_002418920.1 | Thielavia terrestris | XP_003653121 |
| Myceliophthora | XP_003660453.1 | | |
| thermophila | | | |

A consensus sequence of MTHFR proteins among land plants is set forth in SEQ ID NO:12, as follows:

MKVIDKIKEA AAEGKTVFSF EFFPPKTEDG VENLFERMDR
MVAHGPSFCD ITWGAGGSTA DLTLEIANKM QNMICVETMM
HLTCTNMPVE KIDHALETIK SNGIQNVLAL RGDPPHGQDK
FVQVEGGFAC ALDLVQHIRA KYGDYFGITV AGYPEAHPDV
IEADGLATPE AYQKDLAYLK KKVDAGADLI VTQLFYDTDI
FLKFVNDCRQ IGITCPIVPG IMPINNYKGF LRMTGFCKTK
IPAEITAALE PIKDNEEAVK AYGIHLGTEM CKKILAHGIK
TLHLYTLNME KSALAILMNL GLIDESKISR SLPWRPPTNV
FRTKEDVRPI FWANRPKSYI SRTIGWDQYP HGRWGDSRNP
SYGALSDYQF MRPRARDKKL QEEWVVPLKS VEDIQEKFKN
YCLGKLKSSP WSELDGLQPE TKIINEQLVK INSKGFLTIN
SQPAVNGEKS DSPSVGWGGP GGYVYQKAYL EFFCSKEKLD
ALVEKCKAFP SLTYMAVNKG GEWKSNVGQT DVNAVTWGVF
PAKEIIQPTV VDPASFMVWK DEAFEIWSRG WASLYPEGDP
SRKLLEEVKN SYFLVSLVDN DYINGDLFAV FAD

MTHFR proteins according to the present invention are those selected from Table 4 (supra), or sequences that have at least a 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the sequences set forth in Table 4, the consensus sequence of SEQ ID NO:12, or the MTHFR sequence of SEQ ID NO:11. Percent identity as used herein refers to the comparison of the proteins of two plants, plant varieties, or organisms as scored by matching amino acids. Percent identity is determined by comparing a statistically significant number of the amino acids from two plants, plant varieties, or organisms and scoring a match when the same two amino acids are present at a position.

The maize MTHFR protein of SEQ ID NO:11 (supra) is an expression product of the maize bm2 gene having a nucleic acid sequence of SEQ ID NO:13, as follows:

CCCTGTCCCC CGCTCCTGCC TCGCCTCACC ACCATTAGAG
CGCGCGCGCC AAGCTCGTGG TGAGGTTTTC GAGATGAAGG
TTATCGAGAA GATCCTGGAG GCGGCGGGGG ATGGCCGGAC
GGCGTTCTCG TTCGAGTACT TCCCTCCCAA GACGGAGGAG
GGGGTCGAGA ACCTGTTCGA GCGGATGGAC CGCATGGTGG
CGCACGGCCC CTCCTTCTGC GACATCACCT GGGGCGCCGG
GGGATCCACC GCTGACCTTA CCCTCGAAAT CGCCAACCGT
ATGCAGAACA TGGTACGTGT CGTGCGGCTA CCTCGCTCGC
GCTCGATCCA CTGCGCGCGA TCTGATCTGA TCTGTTTCGG
CACCGGGAAG TGTTAGCCTG AGTGCCGGCT TCGGTTAGAT
CTGCCTAGGT GCGGGGTAAT GCGGCGGGGG ACGTCGCGAT
ATGCATTCGT TTTGTTGTGT CGGTAACGGT TATGGCCTTT
GGGTAATTAG ATTTCCTGGA TTGGTTCCGA GCGAGAGGGA
ATGCGTTTGG TCAATGCCGG GATTTTGGGT GGTGGTTGCT
CTGGTTGAGT TCGAATTAGT GTGAGTTTGG GCTAAATTCA
AAAATCGTGT TTATATAATC CAGCTTTAGA CCATGTTTGT
TTACCCTCTA CTCTAGAGTA CATAATCCAG CTTATATAAG
TTGAGAGGTA AACAAACAAC ACAGATTATT AGGTGGATTA
TGTTATCTAG ATACCTGGAT TATGATAATC TATAAGCATG
TCAATAGGTG TTTATATAAT CCATAAGCTG GAATGCTGGA
TTATATAATC CTGGAAGGAA ACAAACAAGG CCTTAATGCT
ATTCACGTCA CGATATGCAT TCGTTTTGTT GTGTCGGTAA
CGGTTATGGC CTTTGGGTAA TTAGATTTCC TGGATTGGTT
CCGAGCGAGA GGGAATGCGT TTGGTCAATG CCGGGATTTT
GGGTGGTGGT TGCTCTGGTT GAGTTCGAAT TAGTGTGAGT
TTGGGCTAAA TTCAAAATTC GTGCCCCACA CTGGAGACCT
GGTGGCGATT ACATTAACTT TATTTTATCA ATTGTTATTG
TGTTCATTCT AATCCTTTTT TGTTTCACAA TTTAGATTGT
TCTTAGTTTA TCAGTTTAAA ACTGATGGGA TGCATGATAC
GGTTATGTTA GGGAGATAAT TGGATTTTTG CCACTCACAA
TGTTGTCTTT GTCTATGAAT TGTGGGTCCA GCTGGGTCTA
TGAAATGTGG GCCTGATGGC AATTTTACAA ACTTGTTTCT
TGCTGATGGC AAATATCAGA ATCTCTCTAT GTTAGGATGG
CATCGTGAGT GCAAAATGGT GGCTTTTGAG CACTAATGCA
TTTTATGTTA TTTATTCACT TGGTACTGCG AGTTTGTCAT
GCGTACAGTG AGTACTGATC ATTGTTAAGA GGAAGTTTGT
GCCTAATGCA CAGAACAGTT TTCTGGAAAC ATTAGTATAA
TCTTGGTTCG ACTTGATCTT CCAGCTTTAG GCTATGTTTG
TTTACCCTCT ACTCTAGAGT ACATAATCCA GCTTATATAA
GTTGAGAGGT AAACAAACAA CACAGATTAT TAGGTGGATT
ATGTTATCTA GATACCTGGA TTCTGATAAT CTATAAGCAT
GTCAATAGGT GTTTATATAA TCCATAAGCT GGATTATATA
ATCCTGGAAG GAAACAAACA GGGCCTTAAT GCTATTCTGT
TCTGAACCGC TTCAATTTTA TTAAACTAAT TTATTTCTTA
TAATTCCTCT GCATCTTTGA TGTATCCCTT GCACCGCATG
CATTAGATGC TTATAATATT TTATATTGGT TCCGTTTTGT
AGGTGTGTGT GGAAACCATG ATGCACCTGA CATGTACAAA
CATGCCAGTA GAGAAGATTG ACCATGCCTT GGAAACCATC
AAATCCAATG GAGAAGATTG GGATTCAAAA TGTTTTGGCC
CTCAGAGGGG ATCCTCCACA TGGGCAAGAC AAATTTGTGC
AAGTTGAAGG CGGATTTGCT TGTGCTCTTG ATTTGGTAAG
ATTGCGTTAA GGGCAACATA AATCGTTGAG TATGTTGGCA
TGACTTGGGC GTGATGACTA GTTTATTGCT GCTGCAATGT
CTGTTATATT TTGGAAACTA GATCAACCCA TCTGAATCCT
CAAAGAAACC ACCTCACATT GAAAGTGTCT TATGTCTTTT
ACTTGCAGGT GCAGCATATT AGAGCCAAGT ACGGTGATTA
TTTTGGCATA ACTGTAGCTG GTTATCCAGG TCAACTAGCC
ATTACATGTT TTTAGAGGGG ATGCTTTCAA GTCCCTACCC
TGTAAATTGG ATGCTTTATC TGAAATAACT TTTTCCAAAT
GTCAGAGGCA CACCCTGATG CGATACAAGG CGAGGGAGGT
GCTACATTGG AAGCATATAG CAATGATCTT GCGTACTTGA
AGAGAAAGGT TCTCTCTACT TTCTTGATTT TGTTTACTTT
TCTCAATTCA AAATGATAGC AGAATACATT TTTGTTGGTT
CATGAACCAA ATAATAAGTT GAAAACGGTA TGATGTTGTA
AGTCATGACA CTTTATATGT TTTGTTCGCT TCTCACTCTG
TATATTTTCT CTGTAGGTTG ATGCTGGTGC TGACCTTATT
GTTACACAAC TTTTCTATGA TACCGACATC TTTCTCAAGT
TTGTGAATGA CTGCCGCCAA ATTGGTATTA CTTGTCCCAT
TGTTCCTGGC ATAATGCCAA TAAATAACTA CAAAGGTTTC
CTGCGCATGA CTGGGTTCTG CAAAACTAAG GTAAGATCTG
TTGCTGGTAG TTCCATCGTT TGTAATTGTT TGGGCGTTTG
GGGATCATTC TATTATCACT TTGGCATGGA CGTATGCTGT
TATGCATATA TTAGCTTTTC ACTTGGCTGA TTTTTTTTAT
CTCAATGCCT GAATTTGAAT GGAGAATAGC TTTCCCACTT
GCGAGTGTAG GTAAATGTAG TGCTAACACC ATATAACATT
CCTCAAGATT AATGAAAAAT TTCAAATTCA CACCATTCTG
TTAATTCACT AATAGTTCAT TGTACATTTA GGTAAATGTT
CTGCAAACAC CATCTCCTTA TAATTTGAAC TTTTATGCCA
TTATGTTAAT GGACAAATAT TTTTGCATTT TTTTCAAATG
CTTTGATTTC TCACTAAAAT AATCACTTTG AATGTTTAAC
AGATACCTTC TGAGATCACT GCTGCACTAG ATCCTATCAA
AGACAATGAG GAGGCTGTTA GACAATATGG AATCCACCTT
GGAACTGAGA TGTGCAAGAA AATTCTTGCT ACTGGCATTA
AGACTTTGCA CCTTTACACA CTAAACATGG ACAAGTCTGC
TATAGGAATT TTGATGGTAA TTTCCATGTC AGAATTTTAT
TTTTTTGTAC CGACCTCTTA CTAACAACTA TTTTGTCCCC
ACCAGAATCT TGGATTAATT GAGGAGTCCA AGGTTTCAAG
GCCATTACCT TGGAGGCCAG CGACTAATGT TTTCCGTGTT
AAAGAGGATG TTCGACCTAT ATTCTGGTAG GTATTGTAGT
TCTTTTATGT TAAGATGCTT GAGGTCTTTG TGACATTCTA
ACCCAGTCTA GTGAATGCTA ATGATTGTGC AGGGCCAACA
GACCAAAGAG CTATCTTAAA AGGACATTAG GTTGGGATCA
GTATCCCCAT GGACGGTGGG GTGATTCTCG GAACCCATCA
TATGGAGCAC TTACTGACCA CCAGGTAATT TCAGTATATC
TGTTCAGGTC TACTTTTTTT TGTGTTTACT GCAATGTTTT
AACGTCACAA ATGCAGTATG CAATATTTTT GTATCATTCT
CAAGGTAATA CAAAATTATA CATTTTCCAG GAAGAAAGGG
TTAAGTTAGA ATGAACATTT AATATGATTC GGAGATATTT
ATTCAGCAAT AAGAAAATAG TATCATGGAC CCCAAATGAG
TGTTCCACTA TAGGATTTCT TCACACTGCC ACTGCTACAC
CTTTATATAG ACTTGATCAA CAAATATATA TTCAAAATTA
CTCAACTTGG CTTCTGTGAT GACTATTTGT CATAAAATCA
TAGTTGTATG CAATCATCTA TTCATTTTAA ACAGTTCACA
AGACCACGAG GCCGTGGTAA GAAGCTCCAA GAAGAAAGGG
CTGTTCCACT GAAATCTGTG GAGGACATTA GTGAGGTAAC
TGTTAAAATA CGTGATACAT TATTGGTCTT CCTGGCATAG
AAGTTGTCAT TTTTTAAAGT CTTTGCTAAC CCTTCTTAAT
TCTGATATTC TCTGTATGCC AGCGCTTCAC AAACTTCTGT
CAAGGGAAAC TCACAAGCAG CCCATGGTCA GAATTGGACG
GTCTTCAACC AGAGACAAAG ATTATCGATG ACCAGTTGGT
GAATATTAAC CAGAAGGGTT TCCTTACAAT TAACAGCCAA
CCTGCTGTAA ATGGAGAGAA ATCCGACTCG CCTACTGTTG
GTAAGTTTAT CGTATTTTGT TTTATAATAG GTGGCCATGG
GTGTTGAAAT ATAAGTGAAT TGCCCACCTT TCCCCATAAG
CTTAAGCTTC TAGGTTACTC AACACTGAGA GCAGTATTTC
AAGCATCTTC CAATTTAACA CAGGGACCAA CCCTTAATGT
CAGCAATCAG GTTTTGTCGG TCTTTAAATC TTAATGTAAG
AATTGTAGTG TTGAGTCAAC ACTCATGGGT ATCCTAGCAA
ACCGGGGCTT GGGTTCACGT CCATTGTAAT TATCAGCATA
GCGCCACATT GTCGGAGAAT CTCTGCTGCA TATATAGCAA
TTCTTGCCTT TCCAGATCAT CTGGAAATGC CTAGGTCGTT
TATGTCAAAT ACTTGCTCGA ACTAGCATGT CAGCCTTTCT
GACCTGTTTA TTTATGCCTG TTCTGTGTCT ATGTCGTTCA
TATCAAATGC TTGCTCGGAT TAGCATGTGA GCCTTTCTGA
CCCCCGTTTA TTTATGTCCT GTTCTGCAGG TTGGGGTGGT
CCTGGAGGCT ACGTTTATCA GAAGGCCTAC CTCGAATTCT
TCTGCGCAAA GGAGAAGTTG GACCAACTAA TTGAGAAGAT
CAAAGCATTC CCTTCTCTCA CTTACATTGC TGTGAACAAG
GATGGAGAAA CATTCTCCAA TATTTCACCG AACGCCGTGA
ATGCTGTCAC GTGGGGTGTT TTCCCTGGCA AGGAGATTAT
CCAGCCTACG GTTGTGGATC ATGCAAGTTT TATGGTTTGG
AAGGACGAAG CATTTGAAAT CTGGACTCGG GGGTGGGGTT
GCATGTTCCC TGAGGGTGAT TCATCGAGGG AGCTACTAGA
GAAGGTAATG TTCACTGCAA ACCTTTAGAC TTAAACATAA
TATACTGTTC CGCATAATAA TGAGCTCCAT TGTAACATTT
TTTAGGAGAT TTGTTCCTAC AGTTAACTGT TATGTTTGTG
GTTCAGTAAT CACTGTTCAC TGCTCTACTG CACTTTCTGC
TAAGAACATA GTGATTTTTT TTTTTTTTGC ATGAACTGCA
AACATCTTGC TGCACTGGTC TTGAACAGCA TCTTTGTTTA
CAGCATTGTT TTTGTAAGAT TAACCTGCGT TTGCAAAATA
CACTAGTGAA ACCAATCTAC ACACATTGTT CATCTTTCAG
AAGTGTATTT TTTTCAAACA GATTCCTTGG CTGCTGACTT
TCAGGTTCAG AAGACCTACT ACCTGGTCAG CCTCGTTGAC
AACGACTACG TCCAGGGGGA CCTGTTTGCT GCCTTCAAGA
TCTGATTATG CTCTCATGGT CTGTATTGTT GTATGGTTGG
ACCCAACAAT ATTCTTGCAT CCCCGACTAC AGGTCTGATC
GCCATGAAAG CTTCTGTTCA TTTGAGCTCG GGGGAGTCCG
CTAGTCTTAT TTTTCTTGAT TTCTCTTGGC TTTCTGGAAC
CATACGAATC AATAAGAAAT GAGCTGTGTT CACTTTCCAG
TTCCTCCGTG CAAGTGGCGC AACGGCCAGG TTCTAGATGT
AAAATTCGTC CTCTAATACA GAGATTGGTA TTGTATGTTA
GTGTAGGATG CGCTGCTCGC ATTGCACTGT TATTGTGCTT
CTGTTTGGTG GAAATAAAGA TTGGTCGAAA TCTGAGGGCC
AGATTGAAAG CCATATAACT GAGGGGATTG GAGGGGCTAA
ATTTTATTTT TTGATTAATT TTAAATAAGA AGGAGAATCT
AGCCCCTCCG GTTTCTCGCT CCCAATCTAG TCCTGAATGT
TCACAAGCCC CCATTTGAAT TTAGCCGGGT AACCCCATTA
ATTAG

The maize bm2 gene of SEQ ID NO:13 (supra) has a coding sequence of SEQ ID NO:14, as follows:

ATGAAGGTTA TCGAGAAGAT CCTGGAGGCG GCGGGGGATG
GCCGGACGGC GTTCTCGTTC GAGTACTTCC CTCCCAAGAC
GGAGGAGGGG GTCGAGAACC TGTTCGAGCG GATGGACCGC
ATGGTGGCGC ACGGCCCCTC CTTCTGCGAC ATCACCTGGG
GCGCCGGGGG ATCCACCGCT GACCTTACCC TCGAAATCGC
CAACCGTATG CAGAACATGG TGTGTGTGGA AACCATGATG
CACCTGACAT GTACAAACAT GCCAGTAGAG AAGATTGACC
ATGCCTTGGA AACCATCAAA TCCAATGGGA TTCAAAATGT
TTTGGCCCTC AGAGGGGATC CTCCACATGG GCAAGACAAA
TTTGTGCAAG TTGAAGGCGG ATTTGCTTGT GCTCTTGATT
TGGTGCAGCA TATTAGAGCC AAGTACGGTG ATTATTTTGG
CATAACTGTA GCTGGTTATC CAGAGGCACA CCCTGATGCG
ATACAAGGCG AGGGAGGTGC TACATTGGAA GCATATAGCA
ATGATCTTGC GTACTTGAAG AGAAAGGTTG ATGCTGGTGC
TGACCTTATT GTTACACAAC TTTTCTATGA TACCGACATC
TTTCTCAAGT TTGTGAATGA CTGCCGCCAA ATTGGTATTA
CTTGTCCCAT TGTTCCTGGC ATAATGCCAA TAAATAACTA
CAAAGGTTTC CTGCGCATGA CTGGGTTCTG CAAAACTAAG
ATACCTTCTG AGATCACTGC TGCACTAGAT CCTATCAAAG
ACAATGAGGA GGCTGTTAGA CAATATGGAA TCCACCTTGG
AACTGAGATG TGCAAGAAAA TTCTTGCTAC TGGCATTAAG
ACTTTGCACC TTTACACACT AAACATGGAC AAGTCTGCTA
TAGGAATTTT GATGAATCTT GGATTAATTG AGGAGTCCAA
GGTTTCAAGG CCATTACCTT GGAGGCCAGC GACTAATGTT
TTCCGTGTTA AAGAGGATGT TCGACCTATA TTCTGGGCCA
ACAGACCAAA GAGCTATCTT AAAAGGACAT TAGGTTGGGA
TCAGTATCCC CATGGACGGT GGGGTGATTC TCGGAACCCA
TCATATGGAG CACTTACTGA CCACCAGTTC ACAAGACCAC
GAGGCCGTGG TAAGAAGCTC CAAGAGGAAT GGGCTGTTCC
ACTGAAATCT GTGGAGGACA TTAGTGAGCG CTTCACAAAC
TTCTGTCAAG GGAAACTCAC AAGCAGCCCA TGGTCAGAAT
TGGACGGTCT TCAACCAGAG ACAAAGATTA TCGATGACCA
GTTGGTGAAT ATTAACCAGA AGGGTTTCCT TACAATTAAC
AGCCAACCTG CTGTAAATGG AGAGAAATCC GACTCGCCTA
CTGTTGGTTG GGGTGGTCCT GGAGGCTACG TTTATCAGAA
GGCCTACCTC GAATTCTTCT GCGCAAAGGA GAAGTTGGAC
CAACTAATTG AGAAGATCAA AGCATTCCCT TCTCTCACTT
ACATTGCTGT GAACAAGGAT GGAGAAACAT TCTCCAATAT
TTCACCGAAC GCCGTGAATG CTGTCACGTG GGGTGTTTTC
CCTGGCAAGG AGATTATCCA GCCTACGGTT GTGGATCATG
CAAGTTTTAT GGTTTGGAAG GACGAAGCAT TTGAAATCTG
GACTCGGGGG TGGGGTTGCA TGTTCCCTGA GGGTGATTCA
TCGAGGGAGC TACTAGAGAA GGTTCAGAAG ACCTACTACC
TGGTCAGCCT CGTTGACAAC GACTACGTCC AGGGGGACCT
GTTTGCTGCC TTCAAGATCT GA

The method of the present invention can be utilized in conjunction with plant cells from a wide variety of plants, as described supra.

The present invention also relates to plants produced by the method of the present invention, described supra.

A further aspect of the present invention relates to a method for altering lignin concentration or composition in a plant. This method involves transforming a plant cell with a nucleic acid molecule encoding an MTHFR protein capable of altering lignin concentration or composition in a plant operably associated with a promoter to obtain a transformed plant cell. Expression of the nucleic acid molecule in the plant cell causes altered lignin concentration or composition relative to a nontransformed plant cell. The method further involves regenerating a plant from the transformed plant cell under conditions effective to alter lignin concentration or composition in the plant.

When altering lignin concentration or composition is referred to herein, it is meant that the amount of lignin in at least one cell, tissue, or area of a plant is lowered or increased compared to that in a wild-type plant, or that the composition of lignin in at least one tissue or area of a plant is altered compared to that in a wild-type plant. Lignin composition refers to the ratio of lignin-type compounds present in a particular tissue or area of a plant.

In one embodiment, MTHFR protein is overexpressed relative to a nontransformed plant cell. In an alternative embodiment, MTHFR protein is underexpressed relative to a nontransformed plant.

When lower lignin concentration levels are desirable, transcriptional or posttranscriptional gene silencing may be used. The method of interfering with endogenous MTHFR protein expression may involve an RNA-based form of gene-silencing known as RNA interference (“RNAi”) (also known more recently as siRNA for short, interfering RNAs). RNAi is a form of post-transcriptional gene silencing (“PTGS”). PTGS is the silencing of an endogenous gene caused by the introduction of a homologous double-stranded RNA (“dsRNA”), transgene, or virus. In PTGS, the transcript of the silenced gene is synthesized, but does not accumulate because it is degraded. RNAi is a specific form of PTGS, in which the gene silencing is induced by the direct introduction of dsRNA. Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), Hammond et al., “Post-Transcriptional Gene Silencing by Double-Stranded RNA,” Nature Rev. Gen. 2:110-119 (Abstract) (2001); Hamilton et al., “A Species of Small Antisense RNA in Posttranscriptional Gene Silencing in Plants,” Science 286:950-952 (Abstract) (1999); Hammond et al., “An RNA-Directed Nuclease Mediates Post-Transcriptional Gene Silencing in Drosophila Cells,” Nature 404:293-298 (2000); Hutvagner et al., “RNAi: Nature Abhors a Double-Strand,” Curr. Opin. Genetics & Development 12:225-232 (2002), which are hereby incorporated by reference in their entirety). In iRNA, the introduction of double stranded RNA (dsRNA) into animal or plant cells leads to the destruction of the endogenous, homologous mRNA, phenocopying a null mutant for that specific gene. In siRNA, the dsRNA is processed to short interfering molecules of 21-, 22- or 23-nucleotide RNAs (siRNA), which are also called “guide RAs,” (Hammond et al., “Post-Transcriptional Gene Silencing by Double-Stranded RNA,” Nature Rev. Gen. 2:110-119 (Abstract) (2001); Sharp, “RNA Interference-2001,” Genes Dev. 15:485-490 (2001); Hutvagner et al., “RNAi: Nature Abhors a Double-Strand,” Curr. Opin. Genetics & Development 12:225-232 (2002), which are hereby incorporated by reference in their entirety) in vivo by the Dicer enzyme, a member of the RNAse III-family of dsRNA-specific ribonucleases (Hutvagner et al., “RNAi: Nature Abhors a Double-Strand,” Curr. Opin. Genetics & Development 12:225-232 (2002); Bernstein et al., “Role for a Bidentate Ribonuclease in the Initiation Step of RNA Interference,” Nature 409:363-366 (2001); Tuschl, T., “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3 (2000); U.S. Pat. No. 6,737,512 to Wu et al., which are hereby incorporated by reference in their entirety). Successive cleavage events degrade the RNA to 19-21 bp duplexes, each with 2-nucleotide 3′ overhangs (Hutvagner et al., “RNAi: Nature Abhors a Double-Strand,” Curr. Opin. Genetics & Development 12:225-232 (2002); Bernstein et al., “Role for a Bidentate Ribonuclease in the Initiation Step of RNA Interference,” Nature 409:363-366 (2001), which are hereby incorporated by reference in their entirety). The siRNAs are incorporated into an effector known as the RNA-induced silencing complex (RISC), which targets the homologous endogenous transcript by base pairing interactions and cleaves the mRNA approximately 12 nucleotides form the 3′ terminus of the siRNA (Hammond et al., “Post-Transcriptional Gene Silencing by Double-Stranded RNA,” Nature Rev. Gen. 2:110-119 (Abstract) (2001); Sharp, “RNA Interference-2001,” Genes Dev. 15:485-490 (2001); Hutvagner et al., “RNAi: Nature Abhors a Double-Strand,” Curr. Opin. Genetics & Development 12:225-232 (2002); Nykanen et al., “ATP Requirements and Small Interfering RNA Structure in the RNA Interference Pathway,” Cell 107:309-321 (2001), which are hereby incorporated by reference in their entirety).

There are several methods for preparing siRNA, including chemical synthesis, in vitro transcription, siRNA expression vectors, and PCR expression cassettes. In one embodiment, dsRNA for the nucleic acid molecule used in the present invention can be generated by transcription in vivo. This involves modifying the nucleic acid molecule for the production of dsRNA, inserting the modified nucleic acid molecule into a suitable expression vector having the appropriate 5′ and 3′ regulatory nucleotide sequences operably linked for transcription and translation, as described supra, and introducing the expression vector having the modified nucleic acid molecule into a suitable host or subject. Using siRNA for gene silencing is a rapidly evolving tool in molecular biology, and guidelines are available in the literature for designing highly effective siRNA targets and making antisense nucleic acid constructs for inhibiting endogenous protein (U.S. Pat. No. 6,737,512 to Wu et al.; Brown et al., “RNA Interference in Mammalian Cell Culture: Design, Execution, and Analysis of the siRNA Effect,” Ambion TechNotes 9(1):3-5 (2002); Sui et al., “A DNA Vector-Based RNAi Technology to Suppress Gene Expression in Mammalian Cells,” Proc. Nat'l. Acad. Sci. USA 99(8):5515-5520 (2002); Yu et al., “RNA Interference by Expression of Short-Interfering RNAs and Hairpin RNAs in Mammalian Cells,” Proc. Nat'l. Acad. Sci. USA 99(9):6047-6052 (2002); Paul et al., “Effective Expression of Small Interfering RNA in Human Cells,” Nature Biotechnology 20:505-508 (2002); Brummelkamp et al., “A System for Stable Expression of Short Interfering RNAs in Mammalian Cells,” Science 296:550-553 (2002), which are hereby incorporated by reference in their entirety). There are also commercially available sources for custom-made siRNAs.

The present invention also relates to a method of making a mutant plant having an altered level of MTHFR protein compared to that of a nonmutant plant. The mutant plant displays an altered lignin concentration or composition phenotype relative to a nonmutant plant. This method involves providing at least one cell of a nonmutant plant containing a gene encoding a functional MTHFR protein and treating the at least one cell of a nonmutant plant under conditions effective to inactivate or overactivate the gene, thereby yielding at least one mutant plant cell containing an inactivate or overactive MTHFR gene. The method further involves propagating the at least one mutant plant cell into a mutant plant. The mutant plant has an altered level of MTHFR protein compared to that of the nonmutant plant and displays an altered lignin concentration or composition phenotype relative to a nonmutant plant.

The functional MTHFR protein can be any MTHFR protein from any of the sources described herein, or any protein with at least 60%, 65%, 70%, 75%, 80%, 85%, 90%, or 95% homology to an MTHFR protein described herein.

In one embodiment, the treating step involves subjecting the at least one cell of the nonmutant plant to a chemical mutagenizing agent under conditions effective to yield at least one mutant plant cell containing an inactive or partially inactive bm2 gene. A suitable chemical mutagenizing agent can include, for example, ethylmethanesulfonate.

In another embodiment, the treating step involves subjecting the at least one cell of the nonmutant plant to a radiation source under conditions effective to yield at least one mutant plant cell containing an inactive bm2 gene. Suitable radiation sources can include, for example, sources that are effective in producing ultraviolet rays, gamma rays, or fast neutrons.

In another embodiment, the treating step involves inserting an inactivating nucleic acid molecule into the gene encoding the functional MTHFR protein or its promoter under conditions effective to inactivate the gene. Suitable inactivating nucleic acid molecules can include, for example, a transposable element. Examples of such transposable elements include, but are not limited to, an Activator (Ac) transposon, a Dissociator (Ds) transposon, or a Mutator (Mu) transposon.

In yet another embodiment of this method of making a mutant plant, the treating step involves subjecting the at least one cell of the nonmutant plant to Agrobacterium transformation under conditions effective to insert an Agrobacterium T-DNA sequence into the gene, thereby inactivating the gene. Suitable Agrobacterium T-DNA sequences can include, for example, those sequences that are carried on a binary transformation vector of pAC106, pAC161, pGABI1, pADIS1, pCSA110, pDAP101, derivatives of pBIN19, or pCAMBIA plasmid series.

In yet another embodiment, the treating step involves subjecting the at least one cell of the nonmutant plant to site-directed mutagenesis of the bm2 gene or its promoter under conditions effective to yield at least one mutant plant cell containing an inactive bm2 gene. See, e.g., Baker, “Gene-editing Nucleases,” Nature Methods 9(1):23-26 (2012), which is hereby incorporated by reference in its entirety. The treating step can also involve mutagenesis by homologous recombination of the bm2 gene or its promoter, targeted deletion of a portion of the bm2 gene sequence or its promoter, and/or targeted insertion of a nucleic acid sequence into the bm2 gene or its promoter. The various plants that can be used in this method are the same as those described supra with respect to the transgenic plants and mutant plants.

Other aspects of the present invention relate to mutant plants produced by this method, as well as mutant plant seeds produced by growing the mutant plant under conditions effective to cause the mutant plant to produce seed.

The present invention also relates to a method for altering lignin concentration or composition in a plant. This method involves transforming a plant cell with a nucleic acid molecule encoding a MTHFR protein capable of determining lignin concentration or composition in a plant operably associated with a promoter to obtain a transformed plant cell. A plant is regenerated from the transformed plant cell. The promoter is induced under conditions effective to alter lignin concentration or composition in the plant.

This method can be utilized in conjunction with plant cells from a wide variety of plants, as described supra. Preferably, this method is used to cause decreased or increased lignin concentration, or altered lignin composition, in maize. The present invention also relates to plants produced by this method of the present invention.

Another aspect of the present invention relates to a method of identifying a candidate plant suitable for breeding that displays an altered lignin concentration or composition phenotype. This method involves analyzing the candidate plant for the presence, in its genome, of an inactive or overactive bm2 gene.

In one embodiment, the method identifies a candidate plant suitable for breeding that displays a decreased lignin concentration or altered lignin composition phenotype. In another embodiment, the method identifies a candidate plant suitable for breeding that displays an increased lignin concentration or altered lignin composition phenotype. Because, as discussed in more detail infra, the bm2 gene controls lignin concentration and lignin composition, if any breeding line contains a mutated bm2 gene, this line will display an altered lignin concentration or composition phenotype. If this line is used as a parental line for breeding purposes, the bm2 gene can be used as a molecular marker for selecting progenies that contain the non-functional or hyper-functional or partially functional bm2 gene. Accordingly, the bm2 gene can be used as a molecular marker for breeding agronomic crops with an altered lignin concentration and/or composition.

Another aspect of the present invention relates to a transgenic plant having an altered level of MTHFR protein capable of determining the lignin concentration or composition in a plant compared to that of a nontransgenic plant. The transgenic plant displays an altered lignin concentration or composition phenotype relative to a nontransgenic plant.

In one embodiment of the present invention, the transgenic plant has a reduced level of MTHFR protein and displays a decreased lignin concentration phenotype in at least some tissues. The plant can be transformed with a nucleic acid construct including a nucleic acid molecule configured to silence MTHFR protein expression.

In another embodiment (as described supra), the transgenic plant is transformed with a nucleic acid construct including a nucleic acid molecule that includes a dominant negative mutation and encodes a non-functional MTHFR protein. This construct is suitable in suppression or interference of endogenous mRNA encoding the MTHFR protein.

In yet another embodiment (as described supra), the transgenic plant is transformed with a nucleic acid construct including a nucleic acid molecule that is positioned in the nucleic acid construct to result in suppression or interference of endogenous mRNA encoding the MTHFR protein.

In still another embodiment (as described supra), the transgenic plant is transformed with a nucleic acid construct including a nucleic acid molecule that encodes the MTHFR protein and is in sense orientation.

In a further embodiment (as described supra), the transgenic plant is transformed with a nucleic acid construct including a nucleic acid molecule that is an antisense form of a MTHFR protein encoding nucleic acid molecule.

In another embodiment (as described supra), the transgenic plant is transformed with first and second of the nucleic acid constructs with the first nucleic acid construct encoding the MTHFR protein in sense orientation and the second nucleic acid construct encoding the MTHFR protein in antisense form.

In yet another embodiment (as described supra), the transgenic plant is transformed with a nucleic acid construct including a nucleic acid molecule including a first segment encoding the MTHFR protein, a second segment in an antisense form of a MTHFR protein encoding nucleic acid molecule, and a third segment linking the first and second segments.

In still another embodiment, the transgenic plant has an increased level of MTHFR protein and displays an increase lignin concentration phenotype in at least some tissue. The plant can be transformed with a nucleic acid construct configured to overexpress MTHFR protein. In another embodiment (as described supra), the nucleic acid construct can include a plant specific promoter, such as an inducible plant promoter.

The present invention further relates to seeds produced from the transgenic plant of the present invention.

### EXAMPLES

The following examples are provided to illustrate embodiments of the present invention but are by no means intended to limit its scope.

### Example 1

### Materials and Methods

Genetic Stocks

Mutator-derived stocks originally obtained from Don Robertson (Iowa State University) have been maintained in the Schnable lab for many years. Various stocks carrying the brown midrib2 reference allele (bm2-ref) were ordered from the Maize Genetics COOP Stock Center. These included 90-896-4/895-2 (Schnable Lab: Ac3247), 93-706-5/705 (Ac3246), 93-705-2/706 (Ac3245), and 2000-1695-7/1695-4 (Ac3244). DNA sequencing revealed that these stocks share the same sequence of exons at the MRHFR. The full length of MTHFR cDNA was first amplified by PCR using the forward primer 5′ GTT ATG AAG GTT ATC GAG AAG ATC CTG GAG 3′ (SEQ ID NO:15) (including the start codon) and the reverse primer 5′ TCA GAT CTT GAA GGC AGC AAA CAG G 3′ (SEQ ID NO:16) (including the stop codon). The corresponding DNA fragment was then sequenced by using the forward primers 5′ATG AAG GTT ATC GAG AAG ATC 3′ (SEQ ID NO:17); 5′ GAT GCG ATA CAA GGC GAG GG 3′ (SEQ ID NO:18); 5′ GCG CTT CAC AAA CTT CTG TC 3′ (SEQ ID NO:19), and the reverse primer 5′ GTA AAG GTG CAA AGT CTT AAT GC 3′ (SEQ ID NO:20). Sequence analysis of full-length MTHFR cDNAs amplified from the various sources of bm2 stocks revealed no polymorphisms relative to the bm2-ref allele, suggesting that all of these stocks carry the same mutant allele. The bm2 allele was maintained by outcrossing heterozygous plants to the inbred line B73 once and then selfing.

Mapping Populations

A bm2 mapping population was created by backcrossing homozygous bm2 mutant plants (pollen) on the inbred line B73 (ear) to generate F1 seeds. F1 plants were self pollinated to create F2 seeds for the bm2 RNA-Seq BSA and bm2 fine-mapping experiments (Schnable Lab: Ac3247, 10B-611).

RNA Preparation from BSR-Seq Samples

F2 seeds from a heterozygous individual (Bm2/bm2-ref; Maize Genetics COOP Stock Center, Stock Center ID: 90-896-4/895-2) (Schnable Lab: Ac3247, 10B-32) were grown in a greenhouse under the conditions of 15 hours of light, 80° F. day, 75° F. night, at 30% humidity. The light intensity was approximately 650-800 μmolm⁻²s⁻¹. Leaf tissue samples were collected from 53 26-day old mutant plants (showing brown midrib phenotype) and 53 nonmutant plants (showing the wild-type phenotype). Measuring from the stem, 5.5 cm of leaf tissue was collected from the 2^{nd} youngest leaf. Corresponding midrib tissue samples were pooled for RNA extraction. Total RNA were extracted from tissues using RNeasy Mini kits (Qiagen, Cologn, Germany) by following the manufacturer's protocol and the resulting samples were treated with DNaseI (Qiagen). The quality of RNA samples was analyzed using ˜100 ng of each RNA sample on the Bioanalyer 2100 RNA chip (Agilent Technologies Inc., Santa Clara, Calif.). This QC experiment was performed according to the manufacturer's protocol. mRNA-Seq libraries were constructed using an Illumina RNA-Seq sample preparation kit (Illumina, Inc., San Diego, Calif.) following the manufacturer's protocol. The libraries were sequenced on Illumina Genome Analyzer II at the Iowa State University DNA facility with 75 cycles, resulting in most sequencing reads having a length of ˜75 bp.

BSR-Seq

BSR-Seq was performed using marker data imputed from RNA-Seq reads (BSR-Seq) to map a gene from a population that has no prior markers available. RNA-Seq is a technology that sequences mRNA in the sample. Read counts for each transcript from the RNA-Seq data correspond with the relative amounts of each transcript, which have been shown to be highly accurate and reproducible for quantifying transcript levels (Pepke et al., “A Computation for ChIP-Seq and RNA-Seq Studies,” Nat. Methods 6:522-32 (2009); Shendure et al., “Next-Generation DNA Sequencing,” Nat. Biotechnol. 26:1135-1145 (2008); Tang et al., “mRNA-Seq Whole-Transcriptome Analysis of a Single Cell,” Nat. Methods 6:377-382 (2009); Wang et al., “RNA-Seq: A Revolutionary Tool for Transcriptomics,” Nat. Rev. Genet. 10:57-63 (2009); and Wilhelm et al., “RNA-Seq-Quantitative Measurement of Expression Through Massively Parallel RNA-Sequencing,”Methods 48:249-257 (2009), which are hereby incorporated by reference in their entirety). BSR-Seq is an adaption of the Bulked Segregation Analysis (BSA) method that can rapidly identify genetic markers linked to a genomic region associated with the selected phenotype (Michelmore et al., “Identification of Markers Linked to Disease-Resistance Genes by Bulked Segregant Analysis: A Rapid Method to Detect Markers in Specific Genomic Regions by Using Segregating Populations,” Proc. Nat. Acad. Sci. U.S.A. 88:9828-9832 (1991), which is hereby incorporated by reference in its entirety). Genetic linkage between markers and the causal gene can be determined via quantification of genetic markers in the selected bulks. Based on the quantitative features of RNA-Seq and its allele-specificity (Pastinen, “Genome-Wide Allele-Specific Analysis: Insights into Regulatory Variation,” Nat. Rev. Genet. 11:533-538 (2010), which is hereby incorporated by reference in its entirety), it is possible to perform BSR-Seq. To map the bm2 gene and to understand the effect of the bm2 mutant on the transcriptome, RNA-Seq was conducted on bm2 mutants and their wild-type siblings. After quantifying allele frequency via read counts in RNA-Seq, a Bayesian-based BSR-Seq approach was developed to map bm2. Both the mapping information and transcriptional profiles from RNA-Seq were used to facilitate the bm2 gene cloning.

Trimming and Mapping of RNA-Seq Reads

Prior to alignment to the reference genome, each read was scanned for low quality bases. Nucleotides having PHRED quality values of <15 (out of 40) or error rates of 0.03% were removed using a custom trimming pipeline, with parameters set similarly to those of the defaults for the trimming software, Lucy. Trimmed reads were aligned to the reference genome using GSNAP and uniquely mapped reads (allowing two mismatches every 36 bp and 3 bp tails per 75 bp) were used for subsequent analyses. The read depth of each gene was computed based on the coordinates of mapped and annotated locations of genes in the reference genome.

Direct Transposon (Mu) Tagging

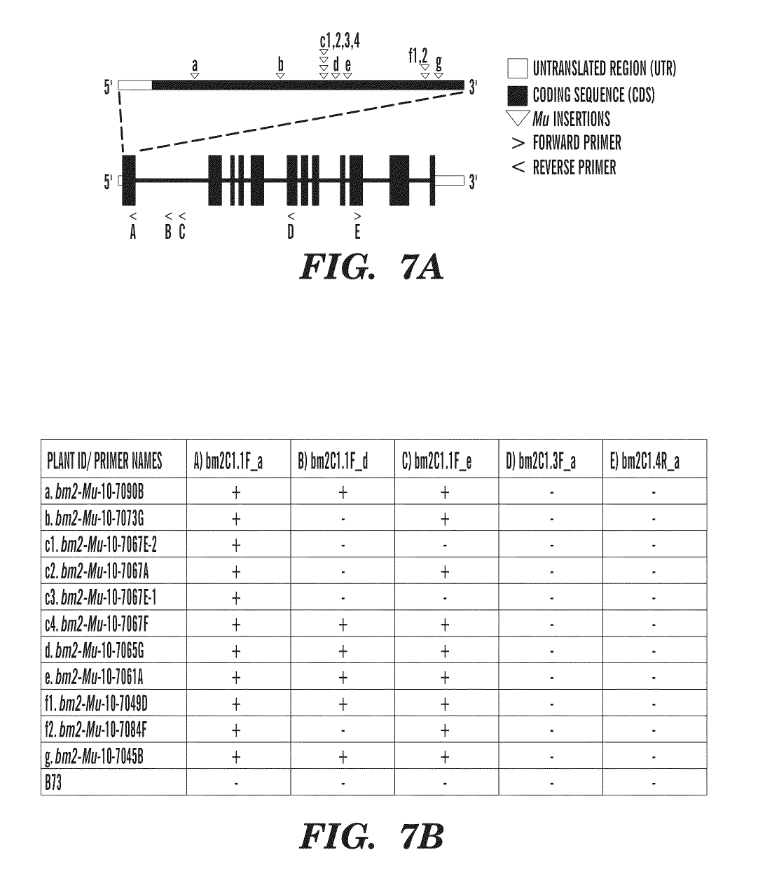

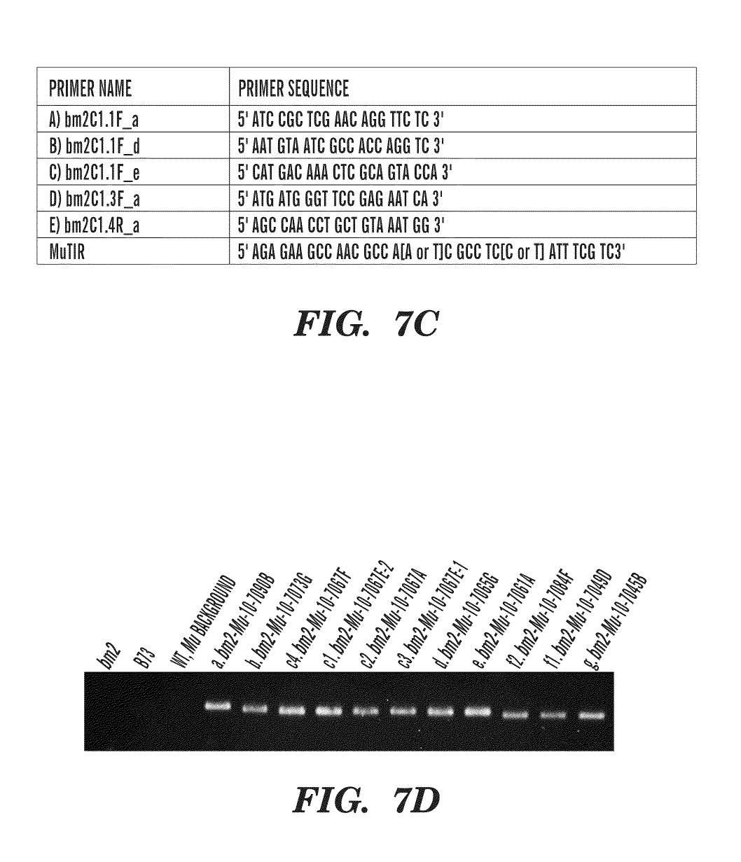

Additional bm2 alleles were identified via a forward genetic screen of a Mutator-derived population. Newly originated bm2-Mu alleles were isolated from around 147,500 progeny of a Mutator-derived population generated by crossing active Mu stocks (Bm2/Bm2; Mu) as females by males homozygous for the bm2-ref allele derived from the four stocks obtained from the Maize Genetics COOP Stock Center 90-896-4/895-2 (Schnable Lab Ac3247), 93-706-5/705 (Ac3246), 93-705-2/706 (Ac3245), and 2000-1695-7/1695-4 (Ac3244). A Mutator transposon specific primer (5′ AGA GAA GCC AAC GCC A[A or T]C GCC TC[C or T] ATT TCG TC 3′ (SEQ ID NO:21)) and a bm2 gene specific primer (5′ ATCCGCTCGAACAGGTTCTC 3′ (SEQ ID NO:22)) were used to analyze rare individuals within the (Bm2/Bm2; Mu×bm2-ref/bm2-ref) population that exhibited a brown midrib phenotype (bm2-Mu/bm2-ref) to identify Mutator insertion alleles in the bm2 locus (FIGS. 7A-D). To generate homozygous bm2-Mu lines, each bm2-ref/bm2-Mu was out-crossed with B73. Five individuals of F2 from each parent were randomly self-crossed. The seeds were germinated for screen of bm2 phenotype, and the F3 that displayed bm2 phenotype were subjected to genotyping for identification of homozygous bm2-Mu line (FIGS. 7A-D).

Phloroglucinol Staining and Light Microscopy

Midrib, stem, and root tissue samples were hand sectioned to a thickness of 200 μm using double-edge razors (Wilkinson Sword, High Wycombe, England), and were temporarily stored in sterile distilled water for 2 hours or less until sample sectioning was completed. Phloroglucinol staining was performed. Briefly, two percent phloroglucinol (Sigma-Aldrich Inc., St. Louis, Mo.) was first dissolved in 95% ethanol (Decon Laboratories, Inc., King of Prussia, Pa.) to form a stock solution. Immediately before use, concentrated hydrochloric acid (33%, v/v) was mixed with the stock solution to form the phloroglucinol stain solution, which was directly applied to samples. Maize tissue samples were placed on glass slides (Thermo Fisher Scientific). Excess solution was removed from the glass slides using Kim Wipes tissues (Kimberly-Clark, Irving, Tex.). 300 μl of phloroglucinol stain was applied on the samples for 30 seconds with a cover glass (Corning, Corning, N.Y.) on top. Additional phloroglucinol staining solution was added to the sample until it was fully covered by the solution. Light images were captured using a Spot RT slider camera (Diagnostic Instruments, Inc., Sterling Heights, Mich.) on a Nikon Eclipse E800 microscope (Nikon, Tokyo, Japan) at 20× magnification, and analyzed using Spot version 4.0.6 software (Diagnostic Instruments, Inc.).

Identification of Differentially Expressed Genes Via Fisher's Exact Test

Normalization was conducted using a method that corrects for biases introduced by RNA composition and differences in the total numbers of uniquely mapped reads in each sample. Normalized read counts were used to calculate fold-changes (FC) and statistical significance. Fisher's exact test was used to test the null hypothesis that expression of a given gene is not different between the two samples. Because this experiment did not include biological replication, statistically significant variation can be a consequence of either biological or technical variation in gene expression between a pair of samples. Genes identified as candidates for differential expression were further filtered with absolute log 2 fold change larger than 1 and a false discovery rate of 0.001% (q-value) to account for multiple testing. These genes were called significantly differentially expressed. Putative maize homologs were functionally classified using the MapMan functional classification system (http://www.ncbi.nlm.nih.gov/pubmed/14996223, which is hereby incorporated by reference in its entirety).

Reverse Transcription Polymerase Chain Reaction (RT-PCR)

Total RNA was isolated and purified by RNeasy Mini Kit (QIAGEN, Cologne, Germany), and 1.5 μg of the total RNA was reverse transcribed into cDNA viaSuperScript™ II Reverse Transcriptase according to manufacturer's instructions (Invitrogen, Carlsbad, Calif.). To detect the level of transcript, PCR cycle parameters used were: 10 minutes at 95° C. (pre-denaturation and hot start), 40 cycles of 35 seconds at 95° C./35 seconds at 58° C./90 seconds at 72° C. (denaturation/annealing/amplification). The following primers were used for detection of their corresponding mRNA. MTHFR forward primer sequence: 5′-ATGAAGGTTATCGAGAAG-3′ (SEQ ID NO:23); reverse primer sequence: 5′-TCAGATCTTGAAGGCAGCAAAC-3′ (SEQ ID NO:24); Actin forward primer sequence: 5′-CCAGGCTGTTCTTTCGTTGT-3′ (SEQ ID NO:25); reverse primer sequence: 5′-CATTAGGTGGTCGGTGAGGT-3′ (SEQ ID NO:26); Cycl forward primer sequence: 5′-CTCCACTACAAGGGCTCCAC-3′ (SEQ ID NO:27); reverse primer sequence: 5′-AACTTCTCGCCGTAGATGGA-3′ (SEQ ID NO:28); Gap forward primer sequence: 5′-GCTTCTCATGGATGGTTGCT-3′ (SEQ ID NO:29); reverse primer sequence: 5′-CAGGAAGGGAAGCAAAAGTG-3′ (SEQ ID NO:30).

Actinomycin D Treatment

A small circular piece of the 2^{nd} youngest leaf (−0.02 g) of various genotypes was obtained using a hole punch. The samples were incubated in water (negative control), actinomycin D (AMD, 50 μg/mL), or DMSO (10 μL/mL, solvent control) for 12 hours at room temperature. Solutions were changed every 4 hours. The samples were then subjected to RNA purification and RT-PCR.

Yeast Complementation Assay

Saccharomyces cerevisiae strains were kindly provided by Dr. Warren D. Kruger (Division of Population Science, Fox Chase Cancer Center, Philadelphia, Pa.) (Shan et al., “Functional Characterization of Human Methylenetetrahydrofolate Reductase in Saccharomyces cerevisiae,” J. Biol. Chem. 274:32613-32618 (1999), which is hereby incorporated by reference in its entirety), with the following genotype: W303-1A (wild-type, also labeled MET11): Mata, ade2-1, can1-100, ura3-1, leu2-3, 112, trp1-1, his3-11, 15; XSY3-1A (Met11 knockout strain, also labeled met11): Mat a, ade2-1, can1-100, ura3-1, leu2-3, 112, trp1-1, his3-11, 15, met11Δ::TRP1. The galactose-inducible human MTHFR expression plasmid (phMTHFR, human MTHFR inserted at phMV2.1) was also provided by Dr. Warren D. Kruger (Shan et al., “Functional Characterization of Human Methylenetetrahydrofolate Reductase in Saccharomyces cerevisiae,” J. Biol. Chem. 274:32613-32618 (1999), which is hereby incorporated by reference in its entirety). To obtain wild-type maize MTHFR cDNA, RNA was extracted from wild-type maize (B73), and then was subjected into RT-PCR. The full length (1782 bp) wild-type maize MTHFR cDNA was first amplified from B73 cDNA using the forward primer 5′ GTT ATG AAG GTT ATC GAG AAG ATC CTG GAG 3′ (SEQ ID NO:31) (including the start codon) and the reverse primer 5′ TCA GAT CTT GAA GGC AGC AAA CAG G 3′ (SEQ ID NO:32) (including the stop codon). The fragment was cloned into the galactose-inducible expression plasmid (pYES2.1/V5-His-TOPO) using pYES2.1 TOPO® TA Expression Kit. Plasmids were transformed to yeast using the S. c. EasyComp™ Transformation Kit (Invitrogen). The cloned fragment was then sequenced by using the forward primers 5′ ATG AAG GTT ATC GAG AAG ATC 3′ (SEQ ID NO:33); 5′ GAT GCG ATA CAA GGC GAG GG 3′ (SEQ ID NO:34); 5′ GCG CTT CAC AAA CTT CTG TC 3′ (SEQ ID NO:35), and the reverse primer 5′ GTA AAG GTG CAA AGT CTT AAT GC 3′ (SEQ ID NO:36). To test the growth of the transformed yeast for complementation, yeast cells were inoculated on SD Glucose-Met (control) plates and SD Galactose-Met (to induce expression of the insert at the plasmid) plates (Clonetech, Mountain View, Calif.; DIFCO, Sparks, Md.), which were then incubated at 30° C. for 3 days.

### Example 2

### Characterization of the bm2 Mutant Phenotype

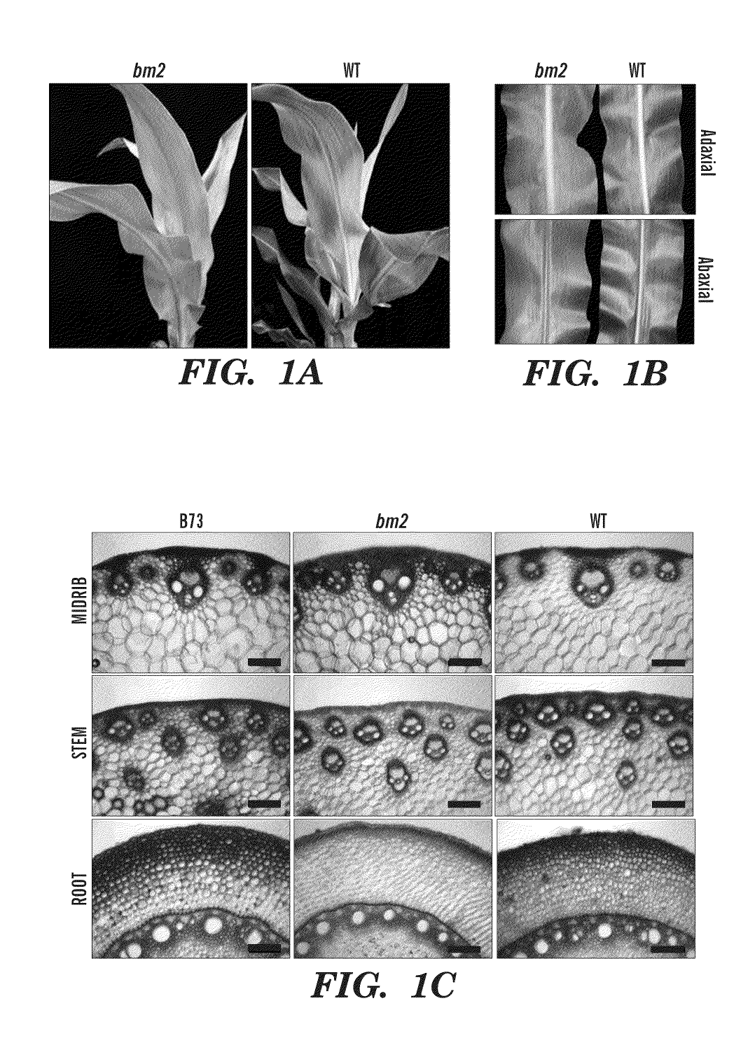

The bm2 mutant was originally identified by its brown pigmentation in the leaf midrib (Neuffer et al., “The Mutants of Maize; A Pictorial Survey in Color of the Usable Mutant Genes in Maize with Gene Symbols and Linkage Map Positions,” Crop Science Society of America Madison, Wis. (1968), which is hereby incorporated by reference in its entirety). This phenotypic description is consistent with observations of the bm2-ref allele. The bm2 mutants in segregating families exhibit a reddish brown pigmentation of the leaf midrib at the 6 to 8 leaf stage, around 27 days after planting (FIG. 1A) (Schnable Lab: Ac3247, 10B-32). The reddish brown pigmentation was observed on both of the adaxial and abaxial surfaces of leaves (FIG. 1B). This pigmentation was not observed in wild-type individuals (FIGS. 1A-B).

Mutant bm2 plants have also been reported to accumulate reduced levels of lignin in their tissues (Sattler et al., “Brown Midrib Mutations and Their Importance to the Utilization of Maize, Sorghum, and Pearl Millet Lignocellulosic Tissues,” Plant Sci. 178:229-238 (2010), which is hereby incorporated by reference in its entirety). This was confirmed in the bm2 mutant tissue samples using microscopy after staining with phloroglucinol (FIG. 1C), which is a traditional method to detect lignin levels in plants (Wardrop, “Lignins: Occurrence, Formation, Structure and Reactions,” Wiley-Interscience, New York (1971), which is hereby incorporated by reference in its entirety). In wild-type maize, the midrib, epidermis, and tissues around vascular bundles stain red with phloroglucinol, thereby demonstrating the lignification of these tissues. In the bm2 mutant, however, these tissues stain only weakly.

Although there are no observable alterations in the anatomy of stems and roots associated with the bm2 mutant, significant differences in phloroglucinol staining were detected in these tissues. In wild-type stems and roots, strong phloroglucinol staining was detected at xylem vessels and epidermis, whereas bm2 mutant tissues exhibited reduced staining, indicating that lignin levels are lower in the bm2 mutant as compared to wild-type controls (FIG. 1C).

### Example 3

### Mapping the bm2 Gene

The focus of this study was to clone and analyze the bm2 gene, which is associated with reductions of lignin content, particularly in G lignin (Sattler et al., “Brown Midrib Mutations and Their Importance to the Utilization of Maize, Sorghum, and Pearl Millet Lignocellulosic Tissues,” Plant Sci. 178:229-238 (2010), which is hereby incorporated by reference in its entirety). The bm2 gene had been previously mapped to chromosome 1 (Neuffer et al., “The Mutants of Maize; A Pictorial Survey in Color of the Usable Mutant Genes in Maize with Gene Symbols and Linkage Map Positions,” Crop Science Society of America Madison, Wis. (1968), which is hereby incorporated by reference in its entirety). To map the bm2 gene to a higher resolution, a modification of Bulked Segregant Analysis (BSA) was used that makes use of RNA-Seq reads. Briefly, RNA-Seq reads are generated from pools of bm2 mutants and wild-type control plants. Because of the digital nature of next-generation sequencing (NGS) data, it is possible to conduct de novo SNP discovery and quantitatively genotype BSA samples using the same RNA-Seq data. In addition, analysis of the RNA-Seq data provides information on the effects of the mutant on global patterns of gene expression at no extra cost.

To generate a bm2 mapping population, a bm2-ref mutant was outcrossed to B73 once to reduce differences in genetic background. An individual heterozygous for the bm2-ref allele was self pollinated to generate an F₂ segregating population. From this segregating population, RNA samples from individuals with the mutant phenotype and nonmutant phenotype were combined into two separate pools (mutant and wild-type) and were subjected to RNA-Seq. To collect tissues for RNA-Seq analysis, midrib tissue was sampled from 53 bm2 mutant individuals and 53 nonmutant individuals (wild-type) 27 days after germination, when the bm2 mutant phenotype first became visible. RNA was extracted from separately pooled tissue samples from bm2 mutants and their wild-type siblings, and then subjected to RNA-Seq. RNA-Seq reads were trimmed and aligned to the reference genome. 46,289 Single Nucleotide Polymorphisms (SNPs) were identified and used for Bulked Segregant Analysis-RNA-Seq (BSR-Seq).

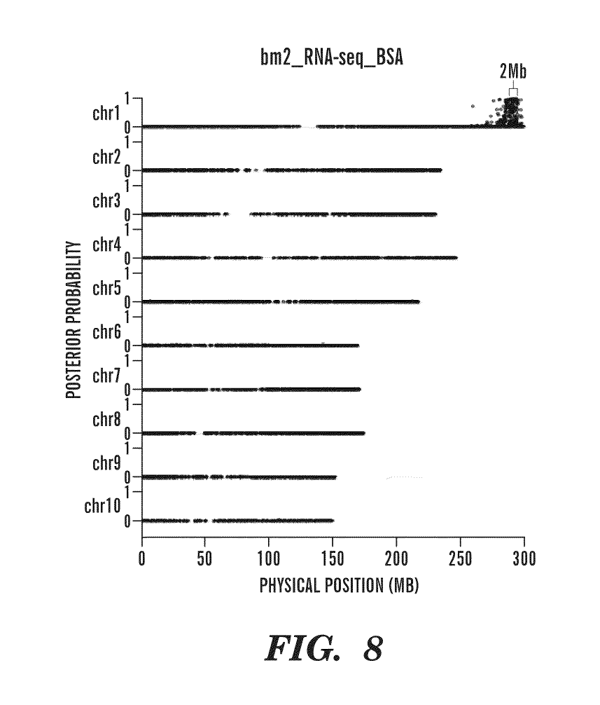

This experiment mapped the bm2 gene to a ˜2 Mb region of Chromosome 1 (FIG. 8). There are 83 genes in the interval of the 2 MB region (Table 1). To narrow down the region of interest, recombinants across this interval were analyzed using SNPs identified from the BSR-Seq experiment by fine mapping (Example 8, infra). This analysis identified the MTHFR gene as a candidate for being the bm2 locus.

TABLE 1
Presence of Genes at the 2 MB Interval as Delineated by BSR-Seq

| GeneID | Chr | Start | End | ExonSize |
|---|---|---|---|---|
| GRMZM2G176774 | 1 | 289685124 | 289690150 | 2844 |
| GRMZM2G168809 | 1 | 289781427 | 289783318 | 875 |
| GRMZM2G168833 | 1 | 289786005 | 289788499 | 1568 |
| GRMZM2G151249 | 1 | 289847066 | 289848058 | 993 |
| GRMZM2G454176 | 1 | 289850950 | 289854988 | 2387 |
| GRMZM2G151266 | 1 | 289862284 | 289873599 | 2142 |
| GRMZM2G029396 | 1 | 290003284 | 290012109 | 2262 |
| GRMZM2G062394 | 1 | 290103711 | 290108581 | 1727 |
| GRMZM2G062377 | 1 | 290108911 | 290111915 | 1960 |
| GRMZM2G062357 | 1 | 290113640 | 290121340 | 2916 |
| GRMZM2G062289 | 1 | 290124962 | 290127148 | 898 |
| GRMZM2G047299 | 1 | 290127887 | 290130698 | 1429 |
| GRMZM2G047223 | 1 | 290130904 | 290135727 | 2080 |
| GRMZM2G349062 | 1 | 290135976 | 290138852 | 1938 |
| GRMZM2G022662 | 1 | 290139770 | 290161386 | 606 |
| GRMZM2G046231 | 1 | 290143390 | 290144830 | 904 |
| GRMZM2G046267 | 1 | 290145735 | 290146996 | 1262 |
| GRMZM2G009901 | 1 | 290200586 | 290203083 | 2498 |
| GRMZM2G170457 | 1 | 290222820 | 290225987 | 1338 |
| GRMZM2G170489 | 1 | 290228650 | 290231967 | 1031 |
| GRMZM2G082664 | 1 | 290265297 | 290286811 | 2095 |
| GRMZM2G432560 | 1 | 290272870 | 290273989 | 1120 |
| GRMZM2G082312 | 1 | 290304861 | 290308869 | 2071 |
| GRMZM2G081943 | 1 | 290316144 | 290318671 | 2163 |
| GRMZM2G382794 | 1 | 290379855 | 290382953 | 573 |
| GRMZM2G161306 | 1 | 290394996 | 290396226 | 1231 |
| GRMZM2G123794 | 1 | 290402922 | 290403710 | 789 |
| GRMZM2G123838 | 1 | 290403982 | 290404661 | 680 |
| GRMZM2G041368 | 1 | 290406956 | 290407513 | 558 |
| GRMZM2G041404 | 1 | 290408543 | 290409351 | 809 |
| GRMZM2G349274 | 1 | 290432647 | 290434899 | 1343 |
| GRMZM2G049432 | 1 | 290435831 | 290436732 | 902 |
| GRMZM2G049370 | 1 | 290436926 | 290438003 | 1078 |
| GRMZM2G013724 | 1 | 290442209 | 290442942 | 734 |
| GRMZM2G013342 | 1 | 290448166 | 290449263 | 1098 |
| GRMZM2G013206 | 1 | 290472057 | 290474246 | 1098 |
| GRMZM2G012224 | 1 | 290521787 | 290523917 | 2040 |
| GRMZM2G312375 | 1 | 290525589 | 290544978 | 963 |
| GRMZM2G092663 | 1 | 290553103 | 290555365 | 1476 |
| GRMZM2G537770 | 1 | 290558691 | 290560434 | 1744 |
| GRMZM2G092746 | 1 | 290561946 | 290563593 | 1437 |
| GRMZM2G092758 | 1 | 290570064 | 290570802 | 343 |
| GRMZM2G111609 | 1 | 2.91E+08 | 2.91E+08 | 1574 |
| GRMZM2G111642 | 1 | 2.91E+08 | 2.91E+08 | 4486 |
| GRMZM2G004511 | 1 | 2.91E+08 | 2.91E+08 | 1443 |
| GRMZM2G004500 | 1 | 2.91E+08 | 2.91E+08 | 1646 |
| GRMZM2G402675 | 1 | 2.91E+08 | 2.91E+08 | 2437 |
| GRMZM2G402714 | 1 | 2.91E+08 | 2.91E+08 | 1016 |
| GRMZM2G104124 | 1 | 2.91E+08 | 2.91E+08 | 705 |
| GRMZM2G104125 | 1 | 2.91E+08 | 2.91E+08 | 2629 |
| GRMZM2G402765 | 1 | 2.91E+08 | 2.91E+08 | 588 |
| GRMZM2G077181 | 1 | 2.91E+08 | 2.91E+08 | 3196 |
| GRMZM2G077079 | 1 | 2.91E+08 | 2.91E+08 | 2700 |
| AC211166.5_FG009 | 1 | 2.91E+08 | 2.91E+08 | 558 |
| GRMZM2G363545 | 1 | 2.91E+08 | 2.91E+08 | 4511 |
| GRMZM2G370275 | 1 | 2.91E+08 | 2.91E+08 | 519 |
| GRMZM2G138340 | 1 | 2.91E+08 | 2.91E+08 | 1135 |
| GRMZM2G138330 | 1 | 2.91E+08 | 2.91E+08 | 1431 |
| GRMZM2G138265 | 1 | 2.91E+08 | 2.91E+08 | 1413 |
| GRMZM2G134227 | 1 | 2.91E+08 | 2.91E+08 | 2250 |
| GRMZM2G064503 | 1 | 2.91E+08 | 2.91E+08 | 2488 |
| GRMZM2G072671 | 1 | 2.91E+08 | 2.91E+08 | 1090 |
| GRMZM2G089266 | 1 | 2.91E+08 | 2.91E+08 | 464 |
| GRMZM2G439654 | 1 | 2.91E+08 | 2.91E+08 | 576 |
| GRMZM2G704127 | 1 | 2.91E+08 | 2.91E+08 | 318 |
| AC197738.3_FG007 | 1 | 2.91E+08 | 2.91E+08 | 429 |
| GRMZM2G116908 | 1 | 2.92E+08 | 2.92E+08 | 2681 |
| GRMZM2G417217 | 1 | 2.92E+08 | 2.92E+08 | 3043 |
| GRMZM2G116878 | 1 | 2.92E+08 | 2.92E+08 | 1063 |
| GRMZM2G704130 | 1 | 2.92E+08 | 2.92E+08 | 371 |
| GRMZM2G179133 | 1 | 2.92E+08 | 2.92E+08 | 1456 |
| GRMZM2G480262 | 1 | 2.92E+08 | 2.92E+08 | 1935 |
| GRMZM2G127404 | 1 | 2.92E+08 | 2.92E+08 | 1360 |
| AC207024.3_FG002 | 1 | 2.92E+08 | 2.92E+08 | 582 |
| GRMZM2G303342 | 1 | 2.92E+08 | 2.92E+08 | 678 |
| GRMZM2G004459 | 1 | 2.92E+08 | 2.92E+08 | 2534 |
| GRMZM2G087612 | 1 | 2.92E+08 | 2.92E+08 | 3469 |
| GRMZM2G704133 | 1 | 2.92E+08 | 2.92E+08 | 924 |
| GRMZM2G087753 | 1 | 2.92E+08 | 2.92E+08 | 3054 |
| GRMZM5G831951 | 1 | 2.92E+08 | 2.92E+08 | 577 |
| GRMZM2G027068 | 1 | 2.92E+08 | 2.92E+08 | 1222 |
| GRMZM2G128663 | 1 | 2.92E+08 | 2.92E+08 | 2538 |
| GRMZM2G107745 | 1 | 2.92E+08 | 2.92E+08 | 917 |

### Example 4

### The MTHFR Gene is Expressed at Lower Levels in the bm2-ref Mutant than in Wild-Type Controls

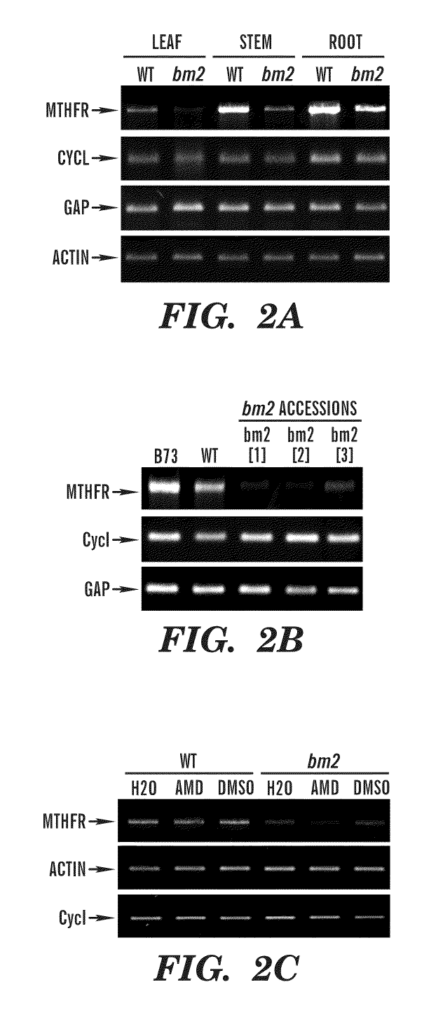

RNA-Seq data from the BSR-Seq experiment suggested that the MTHFR gene is down-regulated in the bm2-ref mutant relative to wild-type controls. Reverse transcription polymerase chain reaction (RT-PCR) experiments confirmed that this pattern holds in multiple tissues (including leaf, stem, and root) (FIG. 2A). Similar results were obtained by analyzing the bm2-ref allele in a variety of genetic backgrounds (FIG. 2B).

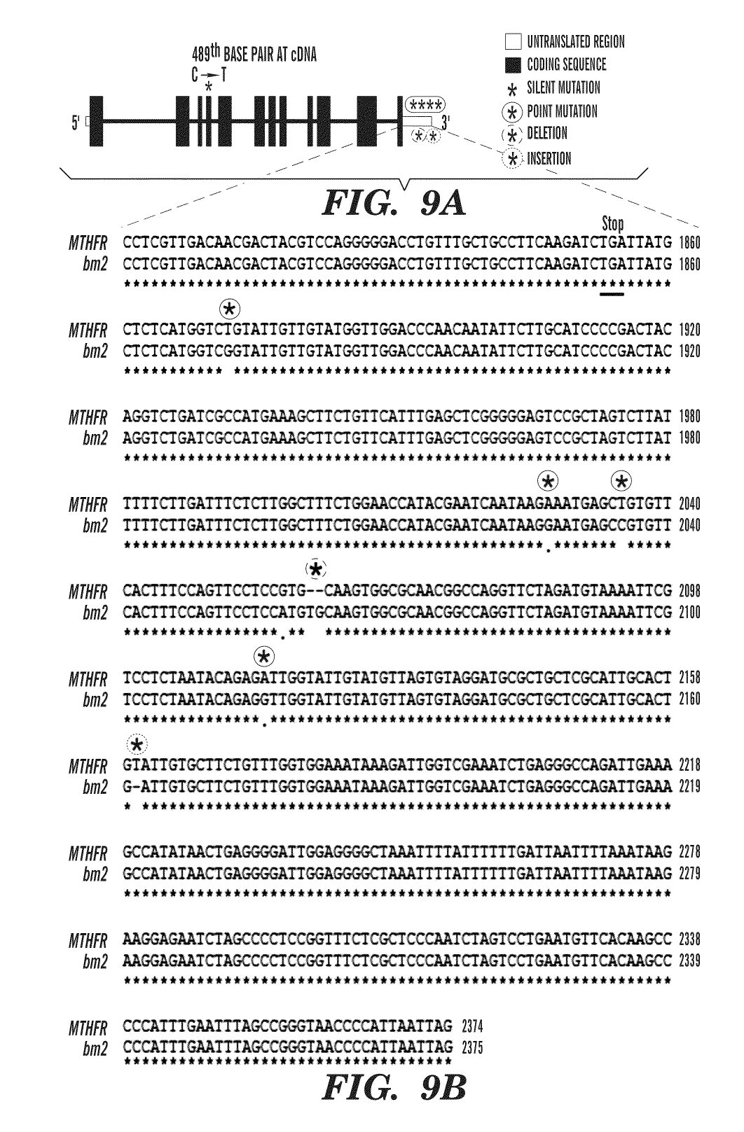

RT-PCR products derived from the MTHFR gene amplified from the bm2-ref mutant were sequenced and compared to the B73 reference genome. Seven polymorphisms were identified in the bm2-ref allele as compared to the B73 reference genome (FIGS. 9A-B). One is a silent mutation in the MTHFR encoding region; the other six polymorphisms are located in the 3′ untranscribed region (UTR) (FIGS. 9A-B). The reduction in the accumulation of MTHFR mRNA in the bm2-ref mutant could be the result of polymorphisms detected between this allele and the B73 wild-type allele (FIGS. 2A-B) because the 3′ UTR plays a critical role in RNA stability (Gutierrez et al., “Current Perspectives on Mrna Stability in Plants: Multiple Levels and Mechanisms of Control,” Trends Plant Sci. 4:429-438 (1999); and Millevoi et al., “Molecular Mechanisms of Eukaryotic Pre-Myna 3′ End Processing Regulation,” Nuc. Acids Res. 38:2757-2774 (2010), which are hereby incorporated by reference in their entirety).

To test the stability of the MTHFR mRNA from wild-type and bm2-ref mutant, the corresponding midrib tissues were exposed to a transcription inhibitor actinomycin D (Sawicki et al., “On the Recovery of Transcription after Inhibition by Actinomycin D,” J. Cell Biol. 55:299-309 (1972), which is hereby incorporated by reference in its entirety). After a 12 hour incubation of the tissues in the presence of actinomycin D, the level of MTHFR mRNA in the bm2-ref tissue was significantly reduced, while the level of the wild-type control remained similar to its original level (FIG. 2C). These results are consistent with the hypothesis that the polymorphisms in the bm2-ref allele reduce the stability of the MRHFR mRNA.

### Example 5

### Confirmation that the bm2 and MTHFR Genes are One-in-the-Same

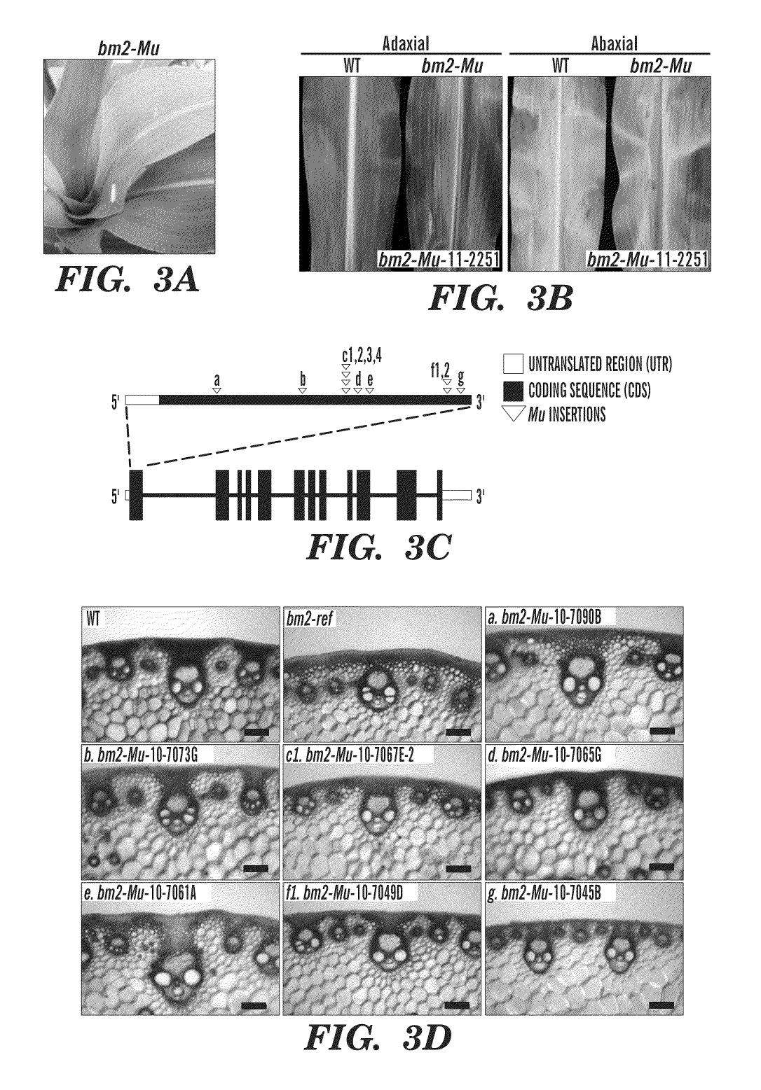

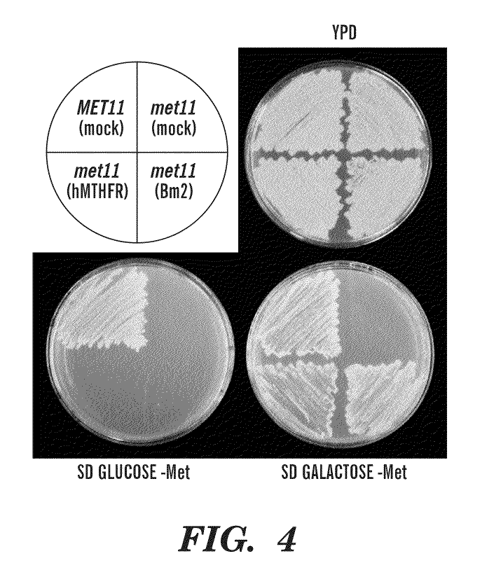

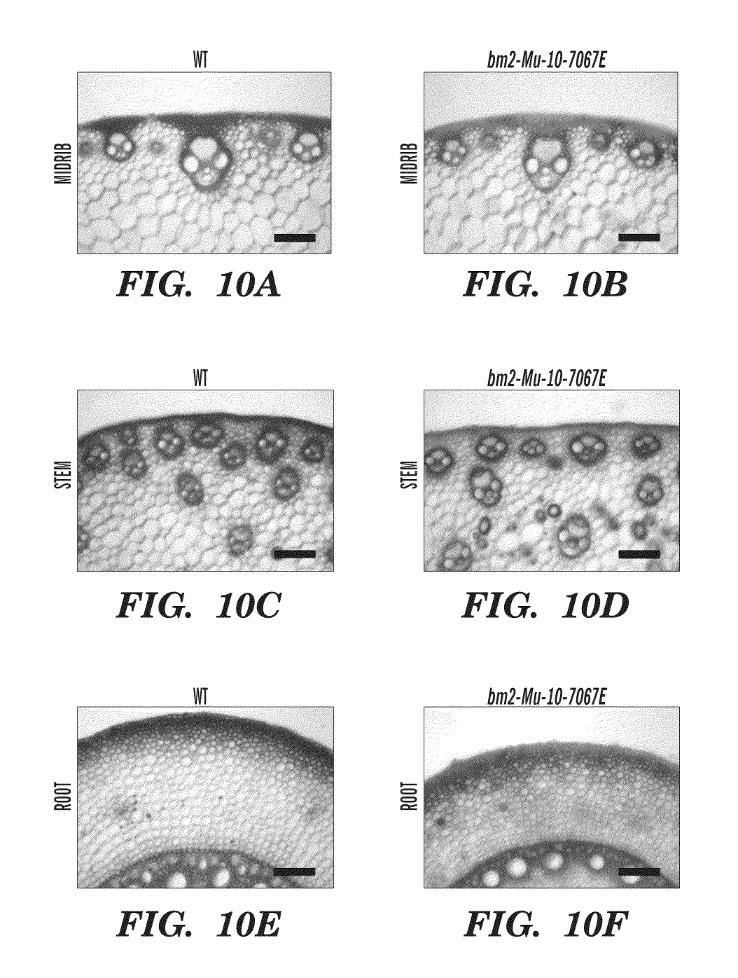

A population of plants derived from a cross of active Mu transposon stocks with the plants homozygous for the bm2-ref allele was screened for novel Mu-induced bm2 mutant alleles. After screening 147,500 individuals, ten plants were identified that exhibited the characteristic reddish-brown midribs of bm2 mutants (FIGS. 3A-B). These individuals were screened using a PCR-based approach with a Mu-specific primer for the terminal-inverted repeat sequence of Mu element together with MTHFR specific primers. Mu insertions were identified in the MTHFR gene in each of the 11 identified mutants, with 6 unique insertion sites (FIG. 3C and FIGS. 7A-D). Homozygous bm2-Mu lines were generated for each of the 11 mutants, and phloroglucinol staining indicated that these individuals accumulate reduced levels of lignin (FIG. 3D, FIGS. 10A-F), demonstrating that the predicted MTHFR gene is indeed the bm2 gene.

### Example 6

### Rescue of MET11 Knockout Yeast by Expression of the MaizeBm2 Gene

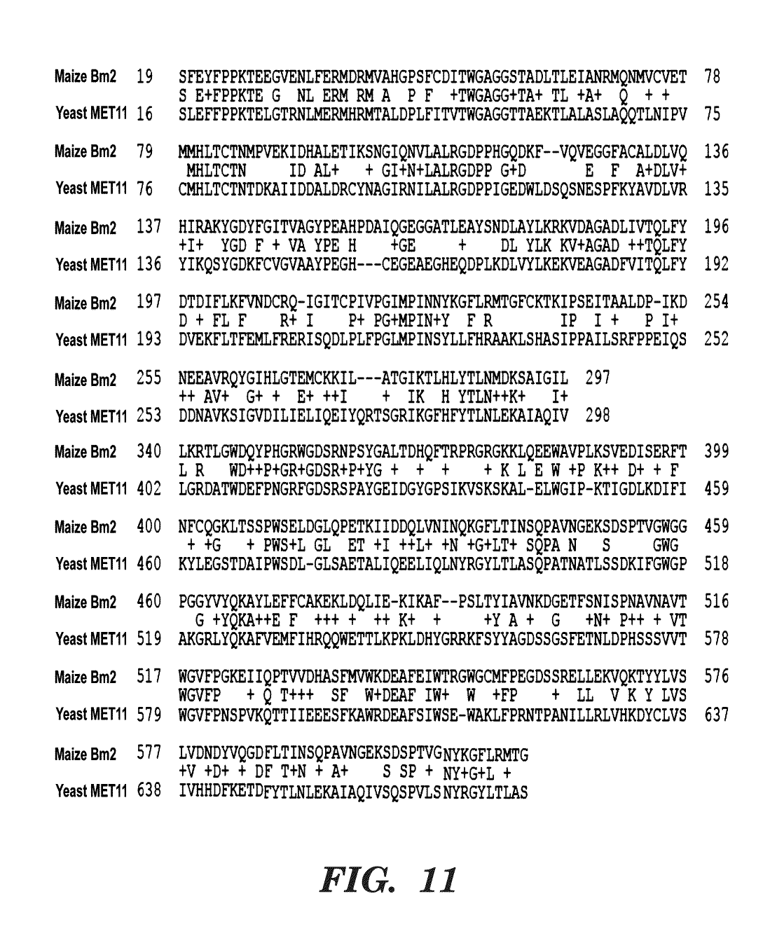

The maize bm2 gene shares 40% of identity and 59% similarity at the amino acid sequence alignment with the yeast MET11 gene, which encodes the enzyme with functional homologue of human MTHFR (FIG. 11). The MTHFR enzyme catalyzes the conversion of 5,10-methylenetetrahydrofolate to 5-methyltetrahydrofolate, a co-substrate for homocysteine remethylation to methionine (Goyette et al., “Human Methylenetetrahydrofolate Reductase Isolation of cDNA, Mapping and Mutation Identification,” Nat. Genet. 7:195-200 (1994); and Vickers et al., “Biochemical and Genetic Analysis of Methylenetetrahydrofolate Reductase in Leishmania Metabolism and Virulence,” J. Biol. Chem. 281:38150-38158 (2006), which (Goyette et al., “Human Methylenetetrahydrofolate Reductase Isolation of cDNA, Mapping and Mutation Identification,” Nat. Genet. 7:195-200 (1994); and Vickers et al., “Biochemical and Genetic Analysis of Methylenetetrahydrofolate Reductase in Leishmania Metabolism and Virulence,” J. Biol. Chem. 281:38150-38158 (2006), which are hereby incorporated by reference in their entirety). Yeast lacking the endogenous MET11 gene can not grow in media lacking methionine (Shan et al., “Functional Characterization of Human Methylenetetrahydrofolate Reductase in Saccharomyces cerevisiae,” J. Biol. Chem. 274:32613-32618 (1999)). To test the hypothesis that the maize bm2 gene encodes MTHFR, a MET11 knockout strain (met11) of yeast was used to test whether expression of the maize bm2 gene could rescue the knockout yeast. As expected, wild-type yeast (MET11), but not the MET11 knockout strain, can grow in the absence of methionine (FIG. 4). Further, consistent with previous reports (Shan et al., “Functional Characterization of Human Methylenetetrahydrofolate Reductase in Saccharomyces cerevisiae,” J. Biol. Chem. 274:32613-32618 (1999), which is hereby incorporated by reference in its entirety), expression of the human MTHFR gene can rescue the MET11 knockout yeast in the present of galactose, which induces the expression of the human MTHFR gene in the construct (FIG. 4) (Shan et al., “Functional Characterization of Human Methylenetetrahydrofolate Reductase in Saccharomyces cerevisiae,” J. Biol. Chem. 274:32613-32618 (1999), which is hereby incorporated by reference in its entirety). When under the control of a galactose-inducible promoter the bm2 cDNA can also rescue MET11 knockout yeast in the present of galactose, but not in the present of glucose, which inhibits the expression of bm2 in this construct (FIG. 4). This finding supports the conclusion that bm2 encodes a functional MTHFR.

### Example 7

### Transcriptomic Analysis of a bm2 Mutant

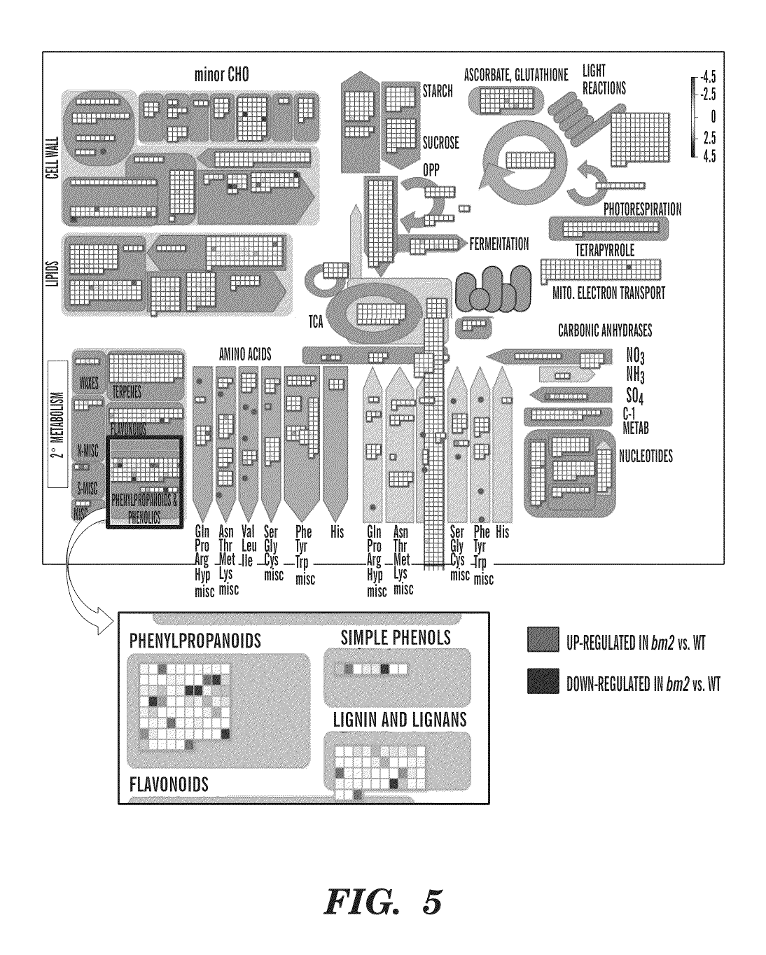

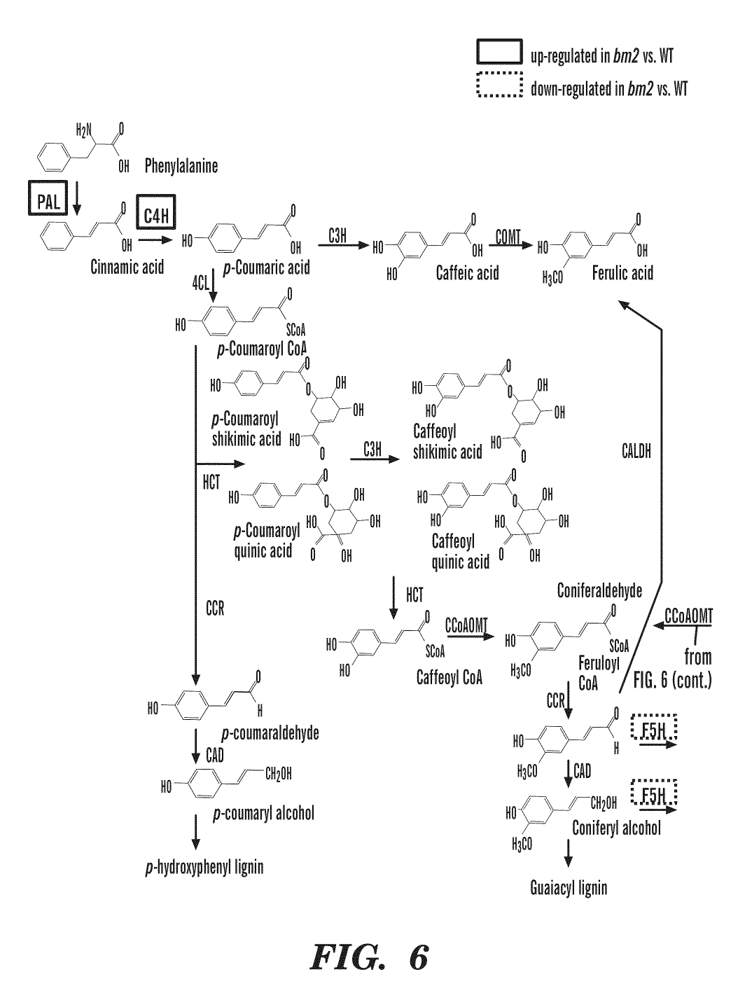

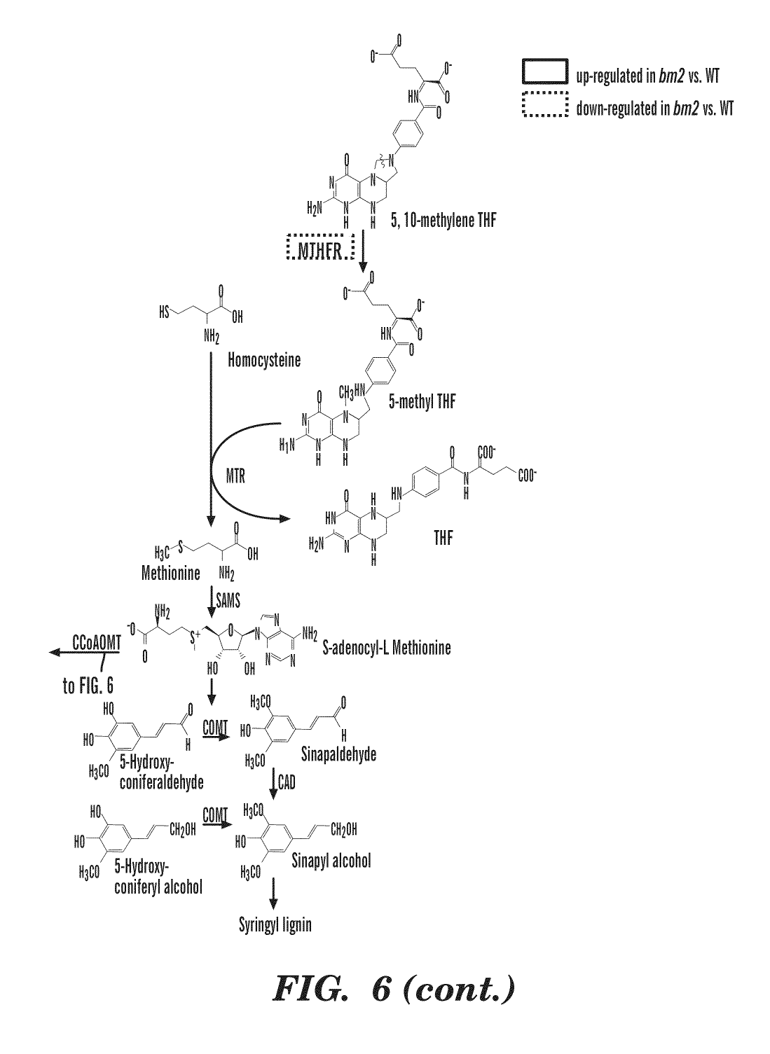

RNA-Seq data from the BSR-Seq experiment were analyzed to compare global gene expression levels in pools of bm2 mutants and wild-type controls. A total of 368 genes were differentially expressed in the bm2 mutant: 242 genes were up-regulated and 126 were down-regulated (FIG. 5). Among the key lignin biosynthetic enzymes, cinnamate 4-hydroxylase (C4H) and phenylalanine ammonia-lyase (PAL) cinnamate 4-hydroxylase (C4H) were up-regulated (Tables 2A-B). In addition to MTHFR, the expression of ferulate 5-hydroxylase (F5H) was reduced (Tables 2A-B), suggesting that methionine biosynthesis may be disturbed in bm2 mutants. Noticeably, no other lignification genes were down-regulated in the bm2 mutants, and no differential expression was observed for cinnamyl alcohol dehydrogenase (CAD), coniferyl aldehyde dehydrogenase (CALDH), Cinnamoyl CoA reductase (CCR); hydroxycinnamoyl CoA: Shikimate hydroxycinnamoyl transferase (HCT); coumaroyl shikimate 3-hydroxylase (C3H); caffeoyl CoA 3-O-methyltransferase (CCoAOMT) or caffeic acid 3-O-methyltransferase (COMT) (FIG. 6 and Table 3).

TABLE 2A
Genes at the 0.51 MB Interval as Narrowed by Fine Mapping
47 genes in the 0.51 MB bm2 interval (working gene set)

| | Gene ID |
|---|---|
| 1 | GRMZM2G179133 |
| 2 | GRMZM2G179133 |
| 3 | GRMZM2G480262 |
| 4 | GRMZM2G590529 |
| 5 | GRMZM2G127397 |
| 6 | GRMZM2G127401 |
| 7 | GRMZM2G558213 |
| 8 | GRMZM2G127404 |
| 9 | AC207024.3_FG002 |
| 10 | GRMZM2G303342 |
| 11 | GRMZM2G004459 |
| 12 | GRMZM2G004459 |
| 13 | GRMZM2G087612 |
| 14 | GRMZM2G388347 |
| 15 | GRMZM2G704133 |
| 16 | GRMZM2G535364 |
| 17 | GRMZM2G087753 |
| 18 | GRMZM2G535366 |
| 19 | GRMZM2G500600 |
| 20 | GRMZM5G831951 |
| 21 | GRMZM2G027068 |
| 22 | GRMZM2G027068 |
| 23 | GRMZM2G500550 |
| 24 | GRMZM2G559608 |
| 25 | GRMZM2G128663 |
| 26 | GRMZM2G128663 |
| 27 | GRMZM2G107745 |
| 28 | GRMZM2G107796 |
| 29 | GRMZM2G039242 |
| 30 | GRMZM2G047365 |
| 31 | GRMZM2G347043 |
| 32 | GRMZM2G347056 |
| 33 | GRMZM2G531215 |
| 34 | GRMZM2G489771 |
| 35 | GRMZM2G009598 |
| 36 | GRMZM2G465553 |
| 37 | GRMZM2G160304 |
| 38 | GRMZM2G160304 |
| 39 | GRMZM2G160304 |
| 40 | GRMZM2G160304 |
| 41 | GRMZM2G160304 |
| 42 | GRMZM2G160304 |
| 43 | GRMZM2G160304 |
| 44 | GRMZM2G160304 |
| 45 | GRMZM2G160304 |
| 46 | GRMZM5G847879 |
| 47 | GRMZM2G072584 |

TABLE 2B
8 genes in the 0.51 MB bm2 interval (filter gene set)

| | Gene ID |
|---|---|
| 1 | GRMZM2G004459 |
| 2 | GRMZM2G087612 |
| 3 | GRMZM2G128663 |
| 4 | GRMZM2G107745 |
| 5 | GRMZM2G047365 |
| 6 | GRMZM2G347043 |
| 7 | GRMZM2G072584 |
| 8 | GRMZM2G009598 |

TABLE 3
The Sequences of bm2 Fine Mapping Primers for KASPar Assay

| Primer name | Primer 1 sequence | Primer 2 sequence | Common primer |
|---|---|---|---|
| bm2- | 5′GAAGGTGACCAAGTT | 5′GAAGGTCGGAGTCAACG | 5′CGTCGAATGTTGTGGTC |
| 289632923 | CATGCTCAGATTTCATC | GATTCAGATTTCATCAAGC | TCTTGGAA3′ (SEQ ID |
| | AAGCCATGTATCTTC3′ | CATGTATCTTG3′ (SEQ | NO: 39) |
| | (SEQ ID NO: 37) | ID NO: 38) | |
| bm2- | 5′GAAGGTGACCAAGTT | 5′GAAGGTCGGAGTCAACG | 5′GCCACGCCAGCCAGGTC |
| 289764777 | CATGCTGACCTCCAGCC | GATTGACCTCCAGCCCGGA | CA3′ (SEQ ID NO: 42) |
| | CGGACGC3′ (SEQ ID | CGG3′ (SEQ ID | |
| | NO: 40) | NO: 41) | |
| bm2- | 5′GAAGGTGACCAAGTT | 5′GAAGGTCGGAGTCAACG | 5′CCATTTTACTTGTTTGC |
| 290199061 | CATGCTAAACAAACCCA | GATTAACAAACCCACCCTG | CACTGTGAGAAA3′ (SEQ |
| | CCCTGGCTTCAA3′ | GCTTCAG3′ (SEQ ID | ID NO: 45) |
| | (SEQ ID NO: 43) | NO: 44) | |
| bm2- | 5′GAAGGTGACCAAGTT | 5′GAAGGTCGGAGTCAACG | 5′CTTATCCGGCTGCTGGC |
| 290203358 | CATGCTGCGGCTATGTT | GATTCTGCGGCTATGTTCT | GCTT3′ (SEQ ID |
| | CTTAGGCGAG3′ (SEQ | TAGGCGAA3′ SEQ ID | NO: 48) |
| | ID NO: 46) | NO: 47) | |
| bm2- | 5′GAAGGTGACCAAGTT | 5′GAAGGTCGGAGTCAACG | 5′GGGGTCGTCGGCGGGCA |
| 290599983 | CATGCTCACAAATCTCT | GATTGCACAAATCTCTCCT | AT3′ (SEQ ID NO: 51) |
| | CCTCCCCTCTC3′ | CCCCTCTT3′ (SEQ ID | |
| | (SEQ ID NO: 49) | NO: 50) | |
| bm2- | 5′GAAGGTGACCAAGTT | 5′GAAGGTCGGAGTCAACG | 5′CGGGCCTGCGGAAGGGG |
| 290898607 | CATGCTCGTACAGCACC | GATTACGTACAGCACCCGG | AA3′ (SEQ ID NO: 54) |
| | CGGTTCACC3′ (SEQ | TTCACT3′ (SEQ ID | |
| | ID NO: 52) | NO: 53) | |
| bm2- | 5′GAAGGTGACCAAGTT | 5′GAAGGTCGGAGTCAACG | 5′GATGGTCGTCGTCACTC |
| 291111263 | CATGCTCCCGACTTTTC | GATTCCCGACTTTTCCTTC | GTCGT3′ (SEQ ID |
| | CTTCCCTTCC3′ (SEQ | CCTTCA3′ (SEQ ID | NO: 57) |
| | ID NO: 55) | NO: 56) | |
| bm2- | 5′GAAGGTGACCAAGTT | 5′GAAGGTCGGAGTCAACG | 5′TCTGCAATTACTATCGC |
| 291118986 | CATGCTACCAGGTTATA | GATTACCAGGTTATAATGA | TAGAGGTTTTATT3′ |
| | ATGATGCCATGGAG3′ | TGCCATGGAC3′ (SEQ | (SEQ ID NO: 60) |
| | (SEQ ID NO: 58) | ID NO: 59) | |
| bm2- | 5′GAAGGTGACCAAGTT | 5′GAAGGTCGGAGTCAACG | 5′CCGATGGCTCTGGCCTG |
| 291119025 | CATGCTCCTCTAGCGAT | GATTACCTCTAGCGATAGT | CGT3′ (SEQ ID |
| | AGTAATTGCAGATAC3′ | AATTGCAGATAA3′ (SEQ | NO: 63) |
| | (SEQ ID NO: 61) | ID NO: 62) | |
| bm2- | 5′GAAGGTGACCAAGTT | 5′GAAGGTCGGAGTCAACG | 5′GAGATGATAGGCTTCGC |
| 291119339 | CATGCTCCACTACCAAC | GATTCCACTACCAACTTCC | TGGCTTT3′ (SEQ ID |
| | TTCCCTGCGG3′(SEQ | CTGCGA3′ (SEQ ID | NO: 66) |
| | ID NO: 64) | NO: 65) | |
| bm2- | 5′GAAGGTGACCAAGTT | 5′GAAGGTCGGAGTCAACG | 5′ATGTATGCAGGCACCAA |
| 291460726 | CATGCTTCCCCAGCAAC | GATTCTTCCCCAGCAACGA | TGCACCAT3′ (SEQ ID |
| | GATCAGGTC3′ (SEQ | TCAGGTT3′ (SEQ ID | NO: 69) |
| | ID NO: 67) | NO: 68) | |
| bm2- | 5′GAAGGTGACCAAGTT | 5′GAAGGTCGGAGTCAACG | 5′GGAAGGCCGCCGGCCTG |
| 291983683 | CATGCTGAACACCTCCC | GATTGAACACCTCCCTGAA | TA3′ (SEQ ID NO: 72) |
| | TGAACTTCTCC3′ | CTTCTCT3′ (SEQ ID | |
| | (SEQ ID NO: 70) | NO: 71) | |

Methionine plays critical roles in fundamental cellular and biochemical processes, including initiation of mRNA translation, synthesis of DNA, RNA and proteins, cell division, and synthesis of cell wall, cell membrane, and chlorophyll (Kwan, “Conditional Alleles in Mice: Practical Considerations for Tissue-Specific Knockouts,” Genesis 32:49-62 (2002), which is hereby incorporated by reference in its entirety). This is consistent with findings from global gene expression analysis, which reveal that the bm2 mutant alters accumulation of transcripts in critical enzymes that participate in photosynthesis, metabolisms, cell division, signaling, stress and transportation (FIG. 5).

### Discussion of Examples 1-7

Plants containing bm2 mutations exhibit reddish brown pigmentation of the leaf midrib, and also reduced levels and altered composition of lignin, which enhances their digestibility. Here, the molecular characterization of the bm2 gene is reported. The bm2 gene was first mapped to a 2 MB interval via BSR-Seq, and 0.51 MB via fine mapping. Multiple independent Mu-induced alleles of the bm2 gene demonstrated that the bm2 gene is a putative MTHFR gene located in this region and that it is down-regulated in the bm2 mutant. Complementation studies conducted in yeast demonstrate that bm2 encodes a functional MTHFR. Follow-up bioinformatic analyses provide mechanistic insights into the link between methionine and lignin biosynthesis.

Survival of bm2 Mutant with MTHFR Mutation

MTHFR plays a critical role in the biosynthesis of methionine (Goyette et al., “Human Methylenetetrahydrofolate Reductase: Isolation of cDNA, Mapping and Mutation Identification,” Nat. Genet. 7:195-200 (1994); and Vickers et al., “Biochemical and Genetic Analysis of Methylenetetrahydrofolate Reductase in Leishmania Metabolism and Virulence,” J. Biol. Chem. 281:38150-38158 (2006), which are hereby incorporated by reference in their entirety), which is an essential, sulfur-containing amino acid. Apart from its nutritional importance and its central role in the initiation of mRNA translation, methionine indirectly regulates various important cellular processes, including biosynthesis of DNA, RNA, protein, lipid, and hormones (Amir, “Current Understanding of the Factors Regulating Methionine Content in Vegetative Tissues of Higher Plants,” Amino Acids 39:917-931 (2010), which is hereby incorporated by reference in its entirety). As methionine is essential in primary and secondary metabolism, it can be expected that mutation of MTHFR could be lethal. Interestingly, bm2 mutant maize has mutations at MTHFR transcript. The survival of the bm2 mutant could be due to several possibilities. Studies showed that plants might absorb methionine, which is released from the degradation of organic matters to the soil (Arshad et al., “Effect of Soil Applied L-Methionine on Growth, Nodulation and Chemical Composition of Albizia lebbeck L,” Plant and Soil 148:129-135 (1993); and Fitzgerald et al., “Availability of Carbon-Bonded Sulfur for Mineralization in Forest Soils,” Can. J. For. Res. 14:839-843 (1984), which are hereby incorporated by reference in their entirety). There could be undiscovered mechanisms involved in methionine in plants. Most importantly, while there is a silent mutation at the MTHFR encoding sequence, 6 mutations occur at the 3′UTR of the MTHFR mRNA transcript at the bm2 mutant. Noticeably, these mutations reduce the stability of the mRNA. Although bm2 mutant has lower level of MTHFR mRNA than the wild-type, the MTHFR biosynthesis at the bm2 mutant is not completely abolished so that the bm2 mutant can survive the MTHFR mutations.

Link Between the bm2 Gene and Guaiacyl (G) and Syringyl (S) Lignin

The MTHFR enzyme catalyzes the conversion of 5,10-methylenetetrahydrofolate to 5-methyltetrahydrofolate, a cosubstrate for homocysteine remethylation to methionine (Goyette et al., “Human Methylenetetrahydrofolate Reductase: Isolation of cDNA, Mapping and Mutation Identification,” Nat. Genet. 7:195-200 (1994); and Vickers et al., “Biochemical and Genetic Analysis of Methylenetetrahydrofolate Reductase in Leishmania Metabolism and Virulence,” J. Biol. Chem. 281:38150-38158 (2006), which are hereby incorporated by reference in their entirety). The bm2 mutant is characterized by reductions in lignin accumulation and alterations in lignin composition, in which G type lignin is significantly reduced (Sattler et al., “Brown Midrib Mutations and Their Importance to the Utilization of Maize, Sorghum, and Pearl Millet Lignocellulosic Tissues,” Plant Sci. 178:229-238 (2010), which is hereby incorporated by reference in its entirety). Gene expression profiles of the lignin biosynthetic pathways show that, while the accumulation of transcripts from the MTRFR gene is reduced, some key lignin biosynthesis enzymes, including Phenylalanine ammonia-lyase (“PAL”) and Cinnamoyl CoA reductase (“CCR”), are actually increased. This up-regulation of PAL and CCR is surprising given the reduction of lignin accumulation in the bm2 mutant. Hence, these results suggest the critical contributions of MTHFR to lignin accumulation. A previous study showed that S-adenosyl-L methionine, which is a downstream product of MTHFR, is consumed by both CCoAOMT and COMT during the biosynthesis of G and S lignin (Ye et al., “An Alternative Methylation Pathway in Lignin Biosynthesis in Zinnia,” Plant Cell 6:1427-1439 (1994), which is hereby incorporated by reference in its entirety). While the methionine pathway is upstream to both the G and S lignin biosynthesis pathway, the S lignin production is not significantly affected in the bm2 mutant (Sattler et al., “Brown Midrib Mutations and Their Importance to the Utilization of Maize, Sorghum, and Pearl Millet Lignocellulosic Tissues,” Plant Sci. 178:229-238 (2010), which is hereby incorporated by reference in its entirety). This suggests that there is also another as yet unidentified pathway to fuel the biosynthesis of S lignin. Together, these results suggest coordination between the regulation of methionine metabolism and lignin biosynthesis (FIG. 6).

MTHFR as a Potential Target in Regulating Lignin Biosynthesis

These studies reveal that mutations at MTHFR result in a reduction in lignin accumulation and alterations in lignin composition. Expression of the bm2 gene can complement MET11 in yeast, indicating its role in methionine synthesis, as human MTHFR (Shan et al., “Functional Characterization of Human Methylenetetrahydrofolate Reductase in Saccharomyces cerevisiae,” J. Biol. Chem. 274:32613-32618 (1999), which is hereby incorporated by reference in its entirety). Together, these suggest that MTHFR, as well as the methionine biosynthesis pathway, is a potential target for engineering crops that accumulate altered lignin. This is in agreement with a previous study that demonstrated that interference of SMAS, an enzyme downstream of MTHFR and that converts methionine to S-adenosyl-L methionine, suppresses lignin biosynthesis (Shen et al., “High Free-Methionine and Decreased Lignin Content Result From a Mutation in the Arabidopsis S-Adenosyl-L-Methionine Synthetase 3 Gene,” Plant J. 29:371-380 (2002), which is hereby incorporated by reference in its entirety). The present invention demonstrates that the normal accumulation of G-lignin, but surprisingly not S-lignin, is MTHFR-dependent.

The brown midrib mutants such as bm1 (CAD), bm2 (MTHFR), and bm3 (COsMT), and also other maize mutants, including CCR1, naturally display reduction of lignin content. Identification of these mutations reveals the molecular mechanisms of the blockage of lignin biosynthesis, and also suggests important targets in alternating lignin composition for forage improvement and bio fuel production. Theoretically, creating double, triple, or different combinations of these mutants might result in further significant reduction of lignin contents. At the same time, however, studies show that reduction of lignin content in biomass, especially in woody plants, could also affect soil structure and fertility, and distort the net carbon storage balance, as the low lignin biomass can be degraded and metabolized into CO₂ and water by soil micro-organisms rapidly than the high lignin biomass (James et al., “Environmental Effects of Genetically Engineered Woody Biomass Crops,” Biomass. Bioenerg. 14:403-414 (1998), which is hereby incorporated by reference in its entirety). Inversely, up-regulation of genes for lignin biosynthesis might increase the lignin content in plants.

### Example 8

### Fine Mapping

By the newly developed adaptation of RNA-Seq based bulked segregant analysis (BSR-Seq) described herein, the bm2 gene was mapped to an interval of 289-291 MB on chromosome 1. There are 83 genes in the interval of the 2 MB region (Table 1). To search the bm2 gene in this region, fine mapping was performed as reported here.

Identification of Primers for Fine Mapping

Leaf tissue samples of F2 seeds from a heterozygous individual

(Bm2/bm2-ref; Maize Genetics COOP Stock Center, Stock Center ID: 90-896-4/895-2) (Schnable Lab: Ac3247, 10B-32), were collected from 53 26-day old mutant plants (showing brown midrib phenotype) and 53 nonmutant plants (showing the wild-type phenotype), and were then subjected into RNA extraction as described supra. The RNA samples were subjected into Illumina RNA-Seq, resulting in most sequencing reads, which were then aligned between wild-type and mutant samples for SNP callings. The SNP data was sent to KBiosciences (Hoddesdon, UK) for the design of primers for fine mapping.

Identification of Recombinant Plants

The F2 seeds for bm2 fine-mapping experiments (Schnable Lab: Ac3247, 10-5977-6150) were planted. 537 plants germinated. When the plants were at the 4-5 leaf stage, leaf tissue was sampled from each plant in 96-well format plates for DNA isolation. The DNA was subjected for KASPar genotyping using the primers designed by KBiosciences (Table 3).

Results

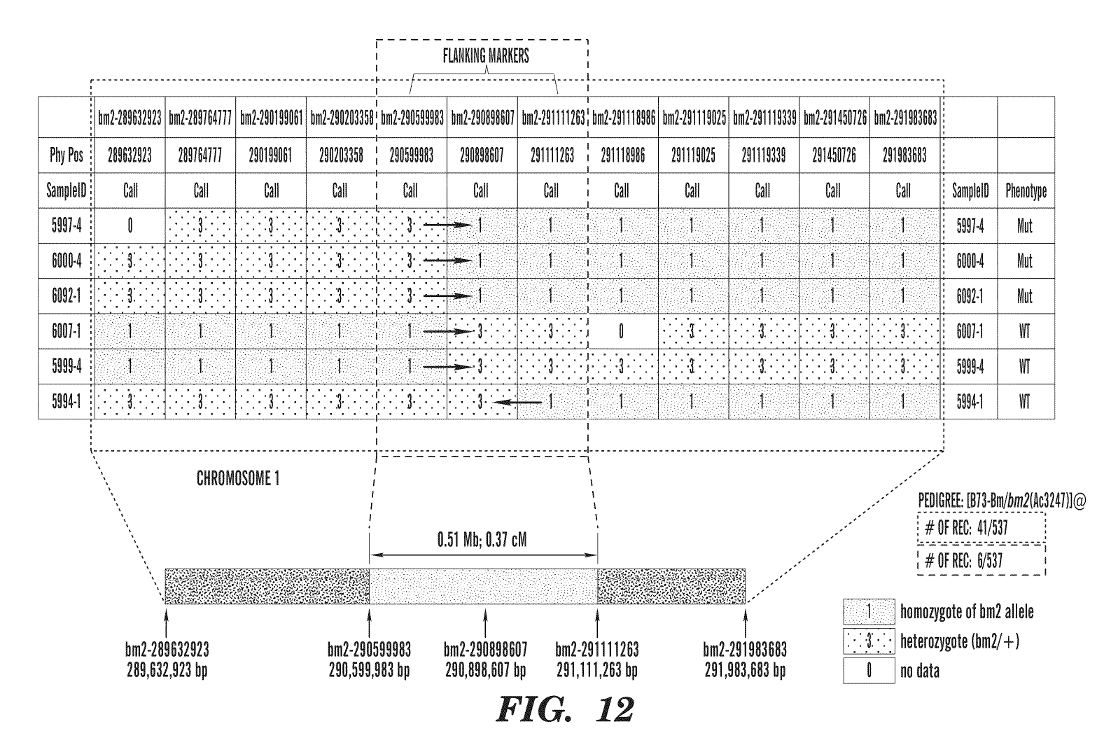

41/537 recombinant plants in the bm2 mapping population were identified within 2 MB interval by using the flanking markers bm2-289632923 and bm2-291983683 (FIG. 12, Table 3). 10 more flanking markers were used to further narrow down the bm2 interval (Table 3). 6/537 recombinant plants were identified within 0.51 MB by using bm2-290599983 and bm2-291111263 (FIG. 12, Table 3). 8 genes (filter gene set) and 47 genes (working gene set) were identified within this interval (Tables 2A-B).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for altering the concentration or composition of lignin in a plant, said method comprising:
providing a transgenic plant or plant seed transformed with a nucleic acid construct effective in altering expression of an MTHFR protein capable of determining the concentration or composition of lignin in a plant; and
growing the transgenic plant or the plant grown from the transgenic plant seed under conditions effective to alter the concentration or composition of lignin in the transgenic plant or the plant grown from the transgenic plant seed.

2. The method according to claim 1, wherein the MTHFR protein has an amino acid sequence that is at least about 80% identical to SEQ ID NO:12.

3. The method according to claim 1, wherein the MTHFR protein has an amino acid sequence that is at least about 90% identical to SEQ ID NO:11.

4. The method according to claim 1, wherein said providing comprises:
providing a nucleic acid construct comprising:
a nucleic acid molecule configured to silence or enhance MTHFR protein expression;
a 5′ DNA promoter sequence; and
a 3′ terminator sequence, wherein the nucleic acid molecule, the promoter, and the terminator are operatively coupled to permit expression of the nucleic acid molecule; and
transforming a plant cell with the nucleic acid construct.

5. The method according to claim 4, wherein the nucleic acid molecule is configured to enhance MTHFR protein expression.

6. The method according to claim 4, wherein the nucleic acid molecule is configured to silence MTHFR protein expression.

7. The method according to claim 6, wherein the nucleic acid molecule comprises a dominant negative mutation and encodes a non-functional MTHFR protein, resulting in suppression or interference of endogenous mRNA encoding the MTHFR protein.

8. The method according to claim 6, wherein the nucleic acid molecule is positioned in the nucleic acid construct to result in suppression or interference of endogenous mRNA encoding the MTHFR protein.

9. The method according to claim 6, wherein the nucleic acid molecule encodes the MTHFR protein and is in sense or antisense orientation.

10. The method according to claim 6, wherein the plant is transformed with first and second of the nucleic acid constructs with the first nucleic acid construct encoding the MTHFR protein in sense orientation and the second nucleic acid construct encoding the MTHFR protein in antisense orientation.

11. The method according to claim 6, wherein the nucleic acid molecule comprises a first segment encoding the MTHFR protein, a second segment in an antisense form of an MTHFR protein encoding nucleic acid molecule, and a third segment linking the first and second segments.

12. A plant produced by the method of claim 1.

13. A method of making a mutant plant having an altered level of MTHFR protein compared to that of a nonmutant plant, wherein the mutant plant displays an altered lignin concentration or composition phenotype relative to a nonmutant plant, said method comprising:
providing at least one cell of a nonmutant plant containing a gene encoding a functional MTHFR protein;
treating said at least one cell of a nonmutant plant under conditions effective to inactivate or overactivate said gene, thereby yielding at least one mutant plant cell containing an inactive or overactive MTHFR gene; and
propagating said at least one mutant plant cell into a mutant plant, wherein said mutant plant has an altered level of MTHFR protein compared to that of the nonmutant plant and displays an altered lignin concentration or composition phenotype relative to a nonmutant plant.

14. A method for altering lignin concentration or composition in a plant, said method comprising:
transforming a plant cell with a nucleic acid molecule encoding an MTHFR protein capable of determining lignin concentration or composition in a plant operably associated with a promoter to obtain a transformed plant cell;
regenerating a plant from the transformed plant cell; and
inducing the promoter under conditions effective to alter lignin concentration or composition in the plant.

15. A method of identifying a candidate plant suitable for breeding that displays an altered lignin concentration or composition phenotype, said method comprising:
analyzing the candidate plant for the presence, in its genome, of an inactive or overactive bm2 gene.

16. The method according to claim 15, wherein the method identifies a candidate plant suitable for breeding that displays an increased lignin concentration and prolonged carbon sequestration phenotype.

17. The method according to claim 15, wherein the method identifies a candidate plant suitable for breeding that displays a decreased lignin concentration phenotype.

18. A transgenic plant having an altered level of MTHFR protein capable of determining the lignin concentration or composition in a plant, compared to that of a nontransgenic plant, wherein the transgenic plant displays an altered lignin concentration or composition phenotype, relative to a nontransgenic plant.

19. The transgenic plant according to claim 18, wherein the transgenic plant has a reduced level of MTHFR protein and displays a decreased concentration of lignan phenotype.

20. The transgenic plant according to claim 19, wherein the plant is transformed with a nucleic acid construct comprising a nucleic acid molecule configured to silence MTHFR protein expression.


SEQUENCE LISTING (72 sequences)

SEQ ID NO: 1 - DNA, 20 bp, artificial
  primer bm2C1.1F_a
atccgctcgaacaggttctc

SEQ ID NO: 2 - DNA, 20 bp, artificial
  primer bm2C1.1F_d
aatgtaatcgccaccaggtc

SEQ ID NO: 3 - DNA, 21 bp, artificial
  primer bm2C1.1F_e
catgacaaactcgcagtacca

SEQ ID NO: 4 - DNA, 20 bp, artificial
  primer bm2C1.3F_a
atgatgggttccgagaatca

SEQ ID NO: 5 - DNA, 20 bp, artificial
  primer bm2C1.4R_a
agccaacctgctgtaaatgg

SEQ ID NO: 6 - DNA, 32 bp, artificial
  primer MuTIR
agagaagccaacgccawcgcctcyatttcgtc

SEQ ID NO: 7 - DNA, 574 bp, Zea mays
cctcgttgacaacgactacgtccagggggacctgtttgctgccttcaagatctgattatgctctcatggtctgtattgttgtatggttggacccaacaatattcttgcatccccgactacaggtctgatcgccatgaaagcttctgttcatttgagctcgggggagtccgctagtcttatttttcttgatttctcttggctttctggaaccatacgaatcaataagaaatgagctgtgttcactttccagttcctccgtgcaagtggcgcaacggccaggttctagatgtaaaattcgtcctctaatacagagattggtattgtatgttagtgtaggatgcgctgctcgcattgcactgtattgtgcttctgtttggtggaaataaagattggtcgaaatctgagggccagattgaaagccatataactgaggggattggaggggctaaattttattttttgattaattttaaataagaaggagaatctagcccctccggtttctcgctcccaatctagtcctgaatgttcacaagcccccatttgaatttagccgggtaaccccattaattag

SEQ ID NO: 8 - DNA, 575 bp, Zea mays
cctcgttgacaacgactacgtccagggggacctgtttgctgccttcaagatctgattatgctctcatggtcggtattgttgtatggttggacccaacaatattcttgcatccccgactacaggtctgatcgccatgaaagcttctgttcatttgagctcgggggagtccgctagtcttatttttcttgatttctcttggctttctggaaccatacgaatcaataaggaatgagccgtgttcactttccagttcctccatgtgcaagtggcgcaacggccaggttctagatgtaaaattcgtcctctaatacagaggttggtattgtatgttagtgtaggatgcgctgctcgcattgcactgattgtgcttctgtttggtggaaataaagattggtcgaaatctgagggccagattgaaagccatataactgaggggattggaggggctaaattttattttttgattaattttaaataagaaggagaatctagcccctccggtttctcgctcccaatctagtcctgaatgttcacaagcccccatttgaatttagccgggtaaccccattaattag

SEQ ID NO: 9 - protein, 557 aa, Zea mays
SFEYFPPKTEEGVENLFERMDRMVAHGPSFCDITWGAGGSTADLTLEIANRMQNMVCVETMMHLTCTNMPVEKIDHALETIKSNGIQNVLALRGDPPHGQDKFVQVEGGFACALDLVQHIRAKYGDYFGITVAGYPEAHPDAIQGEGGATLEAYSNDLAYLKRKVDAGADLIVTQLFYDTDIFLKFVNDCRQIGITCPIVPGIMPINNYKGFLRMTGFCKTKIPSEITAALDPIKDNEEAVRQYGIHLGTEMCKKILATGIKTLHLYTLNMDKSAIGILLKRTLGWDQYPHGRWGDSRNPSYGALTDHQFTRFRGRGKKLQEEWAVPLKSVEDISERFTNFCQGKLTSSPWSELDGLQPETKIIDDQLVNINQKGFLTINSQPAVNGEKSDSPTVGWGGPGGYVYQKAYLEFFCAKEKLDQLIEKIKAFPSLTYIAVNKDGETFSNISPNAVNAVTWGVFPGKEIIQPTVVDHASFMVWKDEAFEIWTRGWGCMFPEGDSSRELLEKVQKTYYLVSLVDNDYVQGDFLTINSQPAVNGEKSDSPTVGNYKGFLRMTG

SEQ ID NO: 10 - protein, 560 aa, Saccharomyces cerevisiae
SLEFFPPKTELGTRNLMERMHRMTALDPLFITVTWGAGGTTAEKTLALASLAQQTLNIPVCMHLTCTNTDKAIIDDALDRCYNAGIRNILALRGDPPIGEDWLDSQSNESPFKYAVDLVRYIKQSYGDKFCVGVAAYPEGHCEGEAEGHEQDPLKDLVYLKEKVEAGADFVITQLFYDVEKFLTFEMLFRERISQDLPLFPGLMPINSYLLFHRAAKLSHASIPPAILSRFPPEIQSDDNAVKSIGVDILIELIQEIYQRTSGRIKGFHFYTLNLEKAIAQIVLGRDATWDEFPNGRFGDSRSPAYGEIDGYGPSIKVSKSKALELWGIPKTIGDLKDIFIKYLEGSTDAIPWSDLGLSAETALIQEELIQLNYRGYLTLASQPATNATLSSDKIFGWGPAKGRLYQKAFVEMFIHRQQWETTLKPKLDHYGRRKFSYYAGDSSGSFETNLDPHSSSVVTWGVFPNSPVKQTTIIEEESFKAWRDEAFSIWSEWAKLFPRNTPANILLRLVHKDYCLVSIVHHDFKETDFYTLNLEKAIAQIVSQSPVLSNYRGYLTLAS

SEQ ID NO: 11 - protein, 593 aa, Zea mays
MKVIEKILEAAGDGRTAFSFEYFPPKTEEGVENLFERMDRMVAHGPSFCDITWGAGGSTADLTLEIANRMQNMVCVETMMHLTCTNMPVEKIDHALETIKSNGIQNVLALRGDPPHGQDKFVQVEGGFACALDLVQHIRAKYGDYFGITVAGYPEAHPDAIQGEGGATLEAYSNDLAYLKRKVDAGADLIVTQLFYDTDIFLKFVNDCRQIGITCPIVPGIMPINNYKGFLRMTGFCKTKIPSEITAALDPIKDNEEAVRQYGIHLGTEMCKKILATGIKTLHLYTLNMDKSAIGILMNLGLIEESKVSRPLPWRPATNVFRVKEDVRPIFWANRPKSYLKRTLGWDQYPHGRWGDSRNPSYGALTDHQFTRPRGRGKKLQEEWAVPLKSVEDISERFTNFCQGKLTSSPWSELDGLQPETKIIDDQLVNINQKGFLTINSQPAVNGEKSDSPTVGWGGPGGYVYQKAYLEFFCAKEKLDQLIEKIKAFPSLTYIAVNKDGETFSNISPNAVNAVTWGVFPGKEIIQPTVVDHASFMVWKDEAFEIWTRGWGCMFPEGDSSRELLEKVQKTYYLVSLVDNDYVQGDLFAAFKI

SEQ ID NO: 12 - protein, 593 aa, artificial
  consensus sequence of MTHFR proteins among land plants
MKVIDKIKEAAAEGKTVFSFEFFPPKTEDGVENLFERMDRMVAHGPSFCDITWGAGGSTADLTLEIANKMQNMICVETMMHLTCTNMPVEKIDHALETIKSNGIQNVLALRGDPPHGQDKFVQVEGGFACALDLVQHIRAKYGDYFGITVAGYPEAHPDVIEADGLATPEAYQKDLAYLKKKVDAGADLIVTQLFYDTDIFLKFVNDCRQIGITCPIVPGIMPINNYKGFLRMTGFCKTKIPAEITAALEPIKDNEEAVKAYGIHLGTEMCKKILAHGIKTLHLYTLNMEKSALAILMNLGLIDESKISRSLPWRPPTNVFRTKEDVRPIFWANRPKSYISRTIGWDQYPHGRWGDSRNPSYGALSDYQFMRPRARDKKLQEEWVVPLKSVEDIQEKFKNYCLGKLKSSPWSELDGLQPETKIINEQLVKINSKGFLTINSQPAVNGEKSDSPSVGWGGPGGYVYQKAYLEFFCSKEKLDALVEKCKAFPSLTYMAVNKGGEWKSNVGQTDVNAVTWGVFPAKEIIQPTVVDPASFMVWKDEAFEIWSRGWASLYPEGDPSRKLLEEVKNSYFLVSLVDNDYINGDLFAVFAD

SEQ ID NO: 13 - DNA, 6275 bp, Zea mays
ccctgtcccccgctcctgcctcgcctcaccaccattagagcgcgcgcgccaagctcgtggtgaggttttcgagatgaaggttatcgagaagatcctggaggcggcgggggatggccggacggcgttctcgttcgagtacttccctcccaagacggaggagggggtcgagaacctgttcgagcggatggaccgcatggtggcgcacggcccctccttctgcgacatcacctggggcgccgggggatccaccgctgaccttaccctcgaaatcgccaaccgtatgcagaacatggtacgtgtcgtgcggctacctcgctcgcgctcgatccactgcgcgcgatctgatctgatctgtttcggcaccgggaagtgttagcctgagtgccggcttcggttagatctgcctaggtgcggggtaatgcgtcggcggacgtcgcgatatgcattcgttttgttgtgtcggtaacggttatggcctttgggtaattagatttcctggattggttccgagcgagagggaatgcgtttggtcaatgccgggattttgggtggtggttgctctggttgagttcgaattagtgtgagtttgggctaaattcaaaaatcgtgtttatataatccagctttagaccatgtttgtttaccctctactctagagtacataatccagcttatataagttgagaggtaaacaaacaacacagattattaggtggattatgttatctagatacctggattatgataatctataagcatgtcaataggtgtttatataatccataagctggaatgctggattatataatcctggaaggaaacaaacaaggccttaatgctattcacgtcacgatatgcattcgttttgttgtgtcggtaacggttatggcctttgggtaattagatttcctggattggttccgagcgagagggaatgcgtttggtcaatgccgggattttgggtggtggttgctctggttgagttcgaattagtgtgagtttgggctaaattcaaaattcgtgccccacactggagacctggtggcgattacattaactttattttatcaattgttattgtgttcattctaatccttttttgtttcacaatttagattgttcttagtttatcagtttaaaactgatgggatgcatgatacggttatgttagggagataattggatttttgccactcacaatgttgtctttgtctatgaattgtgggtccagctgggtctatgaaatgtgggcctgatggcaattttacaaacttgtttcttgctgatggcaaatatcagaatctctctatgttaggatggcatcgtgagtgcaaaatggtggcttttgagcactaatgcattttatgttatttattcacttggtactgcgagtttgtcatgcgtacagtgagtactgatcattgttaagaggaagtttgtgcctaatgcacagaacagttttctggaaacattagtataatcttggttcgacttgatcttccagctttaggctatgtttgtttaccctctactctagagtacataatccagcttatataagttgagaggtaaacaaacaacacagattattaggtggattatgttatctagatacctggattctgataatctataagcatgtcaataggtgtttatataatccataagctggattatataatcctggaaggaaacaaacagggccttaatgctattctgttctgaaccgcttcaattttattaaactaatttatttcttataattcctctgcatctttgatgtatcccttgcaccgcatgcattagatgcttataatattttatattggttccgttttgtaggtgtgtgtggaaaccatgatgcacctgacatgtacaaacatgccagtagagaagattgaccatgccttggaaaccatcaaatccaatgggattcaaaatgttttggccctcagaggggatcctccacatgggcaagacaaatttgtgcaagttgaaggcggatttgcttgtgctcttgatttggtaagattgcgttaagggcaacataaatcgttgagtatgttggcatgacttgggcgtgatgactagtttattgctgctgcaatgtctgttatattttggaaactagatcaacccatctgaatcctcaaagaaaccacctcacattgaaagtgtcttatgtcttttacttgcaggtgcagcatattagagccaagtacggtgattattttggcataactgtagctggttatccaggtcaactagccattacatgtttttagaggggatgctttcaagtccctaccctgtaaattggatgctttatctgaaataactttttccaaatgtcagaggcacaccctgatgcgatacaaggcgagggaggtgctacattggaagcatatagcaatgatcttgcgtacttgaagagaaaggttctctctactttcttgattttgtttacttttctcaattcaaaatgatagcagaatacatttttgttggttcatgaaccaaataataagttgaaaacggtatgatgttgtaagtcatgacactttatatgttttgttcgcttctcactctgtatattttctctgtaggttgatgctggtgctgaccttattgttacacaacttttctatgataccgacatctttctcaagtttgtgaatgactgccgccaaattggtattacttgtcccattgttcctggcataatgccaataaataactacaaaggtttcctgcgcatgactgggttctgcaaaactaaggtaagatctgttgctggtagttccatcgtttgtaattgtttgggcgtttggggatcattctattatcactttggcatggacgtatgctgttatgcatatattagcttttcacttggctgattttttttatctcaatgcctgaatttgaatggagaatagctttcccacttgcgagtgtaggtaaatgtagtgctaacaccatataacattcctcaagattaatgaaaaatttcaaattcacaccattctgttaattcactaatagttcattgtacatttaggtaaatgttctgcaaacaccatctccttataatttgaacttttatgccattatgttaatggacaaatatttttgcatttttttcaaatgctttgatttctcactaaaataatcactttgaatgtttaacagataccttctgagatcactgctgcactagatcctatcaaagacaatgaggaggctgttagacaatatggaatccaccttggaactgagatgtgcaagaaaattcttgctactggcattaagactttgcacctttacacactaaacatggacaagtctgctataggaattttgatggtaatttccatgtcagaattttatttttttgtaccgacctcttactaacaactattttgtccccaccagaatcttggattaattgaggagtccaaggtttcaaggccattaccttggaggccagcgactaatgttttccgtgttaaagaggatgttcgacctatattctggtaggtattgtagttcttttatgttaagatgcttgaggtctttgtgacattctaacccagtctagtgaatgctaatgattgtgcagggccaacagaccaaagagctatcttaaaaggacattaggttgggatcagtatccccatggacggtggggtgattctcggaacccatcatatggagcacttactgaccaccaggtaatttcagtatatctgttcaggtctacttttttttgtgtttactgcaatgttttaacgtcacaaatgcagtatgcaatatttttgtatcattctcaaggtaatacaaaattatacattttccaggaagaaagggttaagttagaatgaacatttaatatgattcggagatatttattcagcaataagaaaatagtatcatggaccccaaatgagtgttccactataggatttcttcacactgccactgctacacctttatatagacttgatcaacaaatatatattcaaaattactcaacttggcttctgtgatgactatttgtcataaaatcatagttgtatgcaatcatctattcattttaaacagttcacaagaccacgaggccgtggtaagaagctccaagaggaatgggctgttccactgaaatctgtggaggacattagtgaggtaactgttaaaatacgtgatacattattggtcttcctggcatagaagttgtcattttttaaagtctttgctaacccttcttaattctgatattctctgtatgccagcgcttcacaaacttctgtcaagggaaactcacaagcagcccatggtcagaattggacggtcttcaaccagagacaaagattatcgatgaccagttggtgaatattaaccagaagggtttccttacaattaacagccaacctgctgtaaatggagagaaatccgactcgcctactgttggtaagtttatcgtattttgttttataataggtggccatgggtgttgaaatataagtgaattgcccacctttccccataagcttaagcttctaggttactcaacactgagagcagtatttcaagcatcttccaatttaacacagggaccaacccttaatgtcagcaatcaggttttgtcggtctttaaatcttaatgtaagaattgtagtgttgagtcaacactcatgggtatcctagcaaaccggggcttgggttcacgtccattgtaattatcagcatagcgccacattgtcggagaatctctgctgcatatatagcaattcttgcctttccagatcatctggaaatgcctaggtcgtttatgtcaaatacttgctcgaactagcatgtcagcctttctgacctgtttatttatgcctgttctgtgtctatgtcgttcatatcaaatgcttgctcggattagcatgtgagcctttctgacccccgtttatttatgtcctgttctgcaggttggggtggtcctggaggctacgtttatcagaaggcctacctcgaattcttctgcgcaaaggagaagttggaccaactaattgagaagatcaaagcattcccttctctcacttacattgctgtgaacaaggatggagaaacattctccaatatttcaccgaacgccgtgaatgctgtcacgtggggtgttttccctggcaaggagattatccagcctacggttgtggatcatgcaagttttatggtttggaaggacgaagcatttgaaatctggactcgggggtggggttgcatgttccctgagggtgattcatcgagggagctactagagaaggtaatgttcactgcaaacctttagacttaaacataatatactgttccgcataataatgagctccattgtaacattttttaggagatttgttcctacagttaactgttatgtttgtggttcagtaatcactgttcactgctctactgcactttctgctaagaacatagtgattttttttttttttgcatgaactgcaaacatcttgctgcactggtcttgaacagcatctttgtttacagcattgtttttgtaagattaacctgcgtttgcaaaatacactagtgaaaccaatctacacacattgttcatctttcagaagtgtatttttttcaaacagattccttggctgctgactttcaggttcagaagacctactacctggtcagcctcgttgacaacgactacgtccagggggacctgtttgctgccttcaagatctgattatgctctcatggtctgtattgttgtatggttggacccaacaatattcttgcatccccgactacaggtctgatcgccatgaaagcttctgttcatttgagctcgggggagtccgctagtcttatttttcttgatttctcttggctttctggaaccatacgaatcaataagaaatgagctgtgttcactttccagttcctccgtgcaagtggcgcaacggccaggttctagatgtaaaattcgtcctctaatacagagattggtattgtatgttagtgtaggatgcgctgctcgcattgcactgttattgtgcttctgtttggtggaaataaagattggtcgaaatctgagggccagattgaaagccatataactgaggggattggaggggctaaattttattttttgattaattttaaataagaaggagaatctagcccctccggtttctcgctcccaatctagtcctgaatgttcacaagcccccatttgaatttagccgggtaaccccattaattag

SEQ ID NO: 14 - DNA, 1782 bp, Zea mays
atgaaggttatcgagaagatcctggaggcggcgggggatggccggacggcgttctcgttcgagtacttccctcccaagacggaggagggggtcgagaacctgttcgagcggatggaccgcatggtggcgcacggcccctccttctgcgacatcacctggggcgccgggggatccaccgctgaccttaccctcgaaatcgccaaccgtatgcagaacatggtgtgtgtggaaaccatgatgcacctgacatgtacaaacatgccagtagagaagattgaccatgccttggaaaccatcaaatccaatgggattcaaaatgttttggccctcagaggggatcctccacatgggcaagacaaatttgtgcaagttgaaggcggatttgcttgtgctcttgatttggtgcagcatattagagccaagtacggtgattattttggcataactgtagctggttatccagaggcacaccctgatgcgatacaaggcgagggaggtgctacattggaagcatatagcaatgatcttgcgtacttgaagagaaaggttgatgctggtgctgaccttattgttacacaacttttctatgataccgacatctttctcaagtttgtgaatgactgccgccaaattggtattacttgtcccattgttcctggcataatgccaataaataactacaaaggtttcctgcgcatgactgggttctgcaaaactaagataccttctgagatcactgctgcactagatcctatcaaagacaatgaggaggctgttagacaatatggaatccaccttggaactgagatgtgcaagaaaattcttgctactggcattaagactttgcacctttacacactaaacatggacaagtctgctataggaattttgatgaatcttggattaattgaggagtccaaggtttcaaggccattaccttggaggccagcgactaatgttttccgtgttaaagaggatgttcgacctatattctgggccaacagaccaaagagctatcttaaaaggacattaggttgggatcagtatccccatggacggtggggtgattctcggaacccatcatatggagcacttactgaccaccagttcacaagaccacgaggccgtggtaagaagctccaagaggaatgggctgttccactgaaatctgtggaggacattagtgagcgcttcacaaacttctgtcaagggaaactcacaagcagcccatggtcagaattggacggtcttcaaccagagacaaagattatcgatgaccagttggtgaatattaaccagaagggtttccttacaattaacagccaacctgctgtaaatggagagaaatccgactcgcctactgttggttggggtggtcctggaggctacgtttatcagaaggcctacctcgaattcttctgcgcaaaggagaagttggaccaactaattgagaagatcaaagcattcccttctctcacttacattgctgtgaacaaggatggagaaacattctccaatatttcaccgaacgccgtgaatgctgtcacgtggggtgttttccctggcaaggagattatccagcctacggttgtggatcatgcaagttttatggtttggaaggacgaagcatttgaaatctggactcgggggtggggttgcatgttccctgagggtgattcatcgagggagctactagagaaggttcagaagacctactacctggtcagcctcgttgacaacgactacgtccagggggacctgtttgctgccttcaagatctga

SEQ ID NO: 15 - DNA, 30 bp, artificial
  forward primer
gttatgaaggttatcgagaagatcctggag

SEQ ID NO: 16 - DNA, 25 bp, artificial
  reverse primer
tcagatcttgaaggcagcaaacagg

SEQ ID NO: 17 - DNA, 21 bp, artificial
  forward primer
atgaaggttatcgagaagatc

SEQ ID NO: 18 - DNA, 20 bp, artificial
  forward primer
gatgcgatacaaggcgaggg

SEQ ID NO: 19 - DNA, 20 bp, artificial
  forward primer
gcgcttcacaaacttctgtc

SEQ ID NO: 20 - DNA, 23 bp, artificial
  reverse primer
gtaaaggtgcaaagtcttaatgc

SEQ ID NO: 21 - DNA, 32 bp, artificial; identical to SEQ ID NO: 6
  Mutator transposon specific primer

SEQ ID NO: 22 - DNA, 20 bp, artificial
  bm2 gene specific primer
atccgctcgaacaggttctc

SEQ ID NO: 23 - DNA, 18 bp, artificial
  MTHFR forward primer
atgaaggttatcgagaag

SEQ ID NO: 24 - DNA, 22 bp, artificial
  MTHFR reverse primer
tcagatcttgaaggcagcaaac

SEQ ID NO: 25 - DNA, 20 bp, artificial
  Actin forward primer
ccaggctgttctttcgttgt

SEQ ID NO: 26 - DNA, 20 bp, artificial
  Actin reverse primer
cattaggtggtcggtgaggt

SEQ ID NO: 27 - DNA, 20 bp, artificial
  Cycl forward primer
ctccactacaagggctccac

SEQ ID NO: 28 - DNA, 20 bp, artificial
  Cycl reverse primer
aacttctcgccgtagatgga

SEQ ID NO: 29 - DNA, 20 bp, artificial
  Gap forward primer
gcttctcatggatggttgct

SEQ ID NO: 30 - DNA, 20 bp, artificial
  Gap reverse primer
caggaagggaagcaaaagtg

SEQ ID NO: 31 - DNA, 30 bp, artificial; identical to SEQ ID NO: 15
  forward primer

SEQ ID NO: 32 - DNA, 25 bp, artificial
  reverse primer
tcagatcttgaaggcagcaaacagg

SEQ ID NO: 33 - DNA, 21 bp, artificial
  forward primer
atgaaggttatcgagaagatc

SEQ ID NO: 34 - DNA, 20 bp, artificial
  forward primer
gatgcgatacaaggcgaggg

SEQ ID NO: 35 - DNA, 20 bp, artificial
  forward primer
gcgcttcacaaacttctgtc

SEQ ID NO: 36 - DNA, 23 bp, artificial
  reverse primer
gtaaaggtgcaaagtcttaatgc

SEQ ID NO: 37 - DNA, 47 bp, artificial
  primer 1 bm2-289632923
gaaggtgaccaagttcatgctcagatttcatcaagccatgtatcttc

SEQ ID NO: 38 - DNA, 47 bp, artificial
  primer 2 bm2-289632923
gaaggtcggagtcaacggattcagatttcatcaagccatgtatcttg

SEQ ID NO: 39 - DNA, 25 bp, artificial
  common primer bm2-289632923
cgtcgaatgttgtggtctcttggaa

SEQ ID NO: 40 - DNA, 39 bp, artificial
  primer 1 bm2-289764777
gaaggtgaccaagttcatgctgacctccagcccggacgc

SEQ ID NO: 41 - DNA, 39 bp, artificial
  primer 2 bm2-289764777
gaaggtcggagtcaacggattgacctccagcccggacgg

SEQ ID NO: 42 - DNA, 19 bp, artificial
  common primer bm2-289764777
gccacgccagccaggtcca

SEQ ID NO: 43 - DNA, 44 bp, artificial
  primer 1 bm2-290199061
gaaggtgaccaagttcatgctaaacaaacccaccctggcttcaa

SEQ ID NO: 44 - DNA, 43 bp, artificial
  primer 2 bm2-290199061
gaaggtcggagtcaacggattaacaaacccaccctggcttcag

SEQ ID NO: 45 - DNA, 29 bp, artificial
  common primer bm2-290199061
ccattttacttgtttgccactgtgagaaa

SEQ ID NO: 46 - DNA, 42 bp, artificial
  primer 1 bm2-290203358
gaaggtgaccaagttcatgctgcggctatgttcttaggcgag

SEQ ID NO: 47 - DNA, 44 bp, artificial
  primer 2 bm2-290203358
gaaggtcggagtcaacggattctgcggctatgttcttaggcgaa

SEQ ID NO: 48 - DNA, 21 bp, artificial
  common primer bm2-290203358
cttatccggctgctggcgctt

SEQ ID NO: 49 - DNA, 43 bp, artificial
  primer 1 bm2-290599983
gaaggtgaccaagttcatgctcacaaatctctcctcccctctc

SEQ ID NO: 50 - DNA, 44 bp, artificial
  primer 2 bm2-290599983
gaaggtcggagtcaacggattgcacaaatctctcctcccctctt

SEQ ID NO: 51 - DNA, 19 bp, artificial
  common primer bm2-290599983
ggggtcgtcggcgggcaat

SEQ ID NO: 52 - DNA, 41 bp, artificial
  primer 1 bm2-290898607
gaaggtgaccaagttcatgctcgtacagcacccggttcacc

SEQ ID NO: 53 - DNA, 42 bp, artificial
  primer 2 bm2-290898607
gaaggtcggagtcaacggattacgtacagcacccggttcact

SEQ ID NO: 54 - DNA, 19 bp, artificial
  common primer bm2-290898607
cgggcctgcggaaggggaa

SEQ ID NO: 55 - DNA, 42 bp, artificial
  primer 1 bm2-291111263
gaaggtgaccaagttcatgctcccgacttttccttcccttcc

SEQ ID NO: 56 - DNA, 42 bp, artificial
  primer 2 bm2-291111263
gaaggtcggagtcaacggattcccgacttttccttcccttca

SEQ ID NO: 57 - DNA, 22 bp, artificial
  common primer bm2-291111263
gatggtcgtcgtcactcgtcgt

SEQ ID NO: 58 - DNA, 46 bp, artificial
  primer 1 bm2-291118986
gaaggtgaccaagttcatgctaccaggttataatgatgccatggag

SEQ ID NO: 59 - DNA, 46 bp, artificial
  primer 2 bm2-291118986
gaaggtcggagtcaacggattaccaggttataatgatgccatggac

SEQ ID NO: 60 - DNA, 30 bp, artificial
  common primer bm2-291118986
tctgcaattactatcgctagaggttttatt

SEQ ID NO: 61 - DNA, 47 bp, artificial
  primer 1 bm2-291119025
gaaggtgaccaagttcatgctcctctagcgatagtaattgcagatac

SEQ ID NO: 62 - DNA, 48 bp, artificial
  primer 2 bm2-291119025
gaaggtcggagtcaacggattacctctagcgatagtaattgcagataa

SEQ ID NO: 63 - DNA, 20 bp, artificial
  common primer bm2-291119025
ccgatggctctggcctgcgt

SEQ ID NO: 64 - DNA, 42 bp, artificial
  primer 1 bm2-291119339
gaaggtgaccaagttcatgctccactaccaacttccctgcgg

SEQ ID NO: 65 - DNA, 42 bp, artificial
  primer 2 bm2-291119339
gaaggtcggagtcaacggattccactaccaacttccctgcga

SEQ ID NO: 66 - DNA, 24 bp, artificial
  common primer bm2-291119339
gagatgataggcttcgctggcttt

SEQ ID NO: 67 - DNA, 41 bp, artificial
  primer 1 bm2-291460726
gaaggtgaccaagttcatgcttccccagcaacgatcaggtc

SEQ ID NO: 68 - DNA, 43 bp, artificial
  primer 2 bm2-291460726
gaaggtcggagtcaacggattcttccccagcaacgatcaggtt

SEQ ID NO: 69 - DNA, 25 bp, artificial
  common primer bm2-291460726
atgtatgcaggcaccaatgcaccat

SEQ ID NO: 70 - DNA, 43 bp, artificial
  primer 1 bm2-291983683
gaaggtgaccaagttcatgctgaacacctccctgaacttctcc

SEQ ID NO: 71 - DNA, 43 bp, artificial
  primer 2 bm2-291983683
gaaggtcggagtcaacggattgaacacctccctgaacttctct

SEQ ID NO: 72 - DNA, 19 bp, artificial
  common primer bm2-291983683
ggaaggccgccggcctgta